SEQUENCE LISTING (35 sequences)

SEQ ID NO: 1 - DNA, 25 bp, Photinus pyralis
ttatgtttttggcgtcttccatggt

SEQ ID NO: 2 - RNA, 19 nt, Photinus pyralis
cuuacgcugaguacuucga

SEQ ID NO: 3 - DNA, 25 bp, Photinus pyralis
cctcttacctcagttacaatttata

SEQ ID NO: 4 - DNA, 21 bp, Photinus pyralis
cuuacgcugaguacuucgatt

SEQ ID NO: 5 - DNA, 21 bp, Photinus pyralis
ucgaaguacucagcguaagtt

SEQ ID NO: 6 - DNA, 21 bp, Escherichia coli
gcgauaagucgugucuuactt

SEQ ID NO: 7 - DNA, 21 bp, Escherichia coli
guaagacacgacuuaucgctt

SEQ ID NO: 8 - DNA, 21 bp, Hepatitis B virus
ucacucaccaaccucuugutt

SEQ ID NO: 9 - DNA, 21 bp, Hepatitis B virus
acaagagguuggugagugatt

SEQ ID NO: 10 - DNA, 20 bp, Mus musculus
cacucagucucuaagggcut

SEQ ID NO: 11 - DNA, 21 bp, Mus musculus
agcccuuagagacugagugtt

SEQ ID NO: 12 - DNA, 21 bp, Aequorea victoria
gaacggcaucaaggugaactt

SEQ ID NO: 13 - DNA, 21 bp, Aequorea victoria
guucaccuugaucccguuctt

SEQ ID NO: 14 - DNA, 21 bp, Hepatitis B virus
ccucuauguaucccuccugtt

SEQ ID NO: 15 - DNA, 21 bp, Hepatitis B virus
caggagggauacauagaggtt

SEQ ID NO: 16 - DNA, 20 bp, Mus musculus
aggcttcgtggattctcttg

SEQ ID NO: 17 - DNA, 21 bp, Mus musculus
cctctatatccgtttccagtc

SEQ ID NO: 18 - DNA, 22 bp, Mus musculus
atcatcttgtgaaacagcagtg

SEQ ID NO: 19 - DNA, 21 bp, Mus musculus
gcatcagcttggacaaggtgt

SEQ ID NO: 20 - DNA, 71 bp, Mus musculus
ggagctttgggaagaggaaggtgtcatccttcctgtcagatggcatcttcctcttcccgaagctccttttt

SEQ ID NO: 21 - DNA, 72 bp, Mus musculus
ggattccaattcagcgggagccacctgatgaagcttgatcgggtggctctcgctgagttggaatccattttt

SEQ ID NO: 22 - DNA, 20 bp, Mus musculus
gggaacagccaccattgttg

SEQ ID NO: 23 - DNA, 20 bp, Mus musculus
gggaagtaaggaccagagac

SEQ ID NO: 24 - DNA, 21 bp, Photinus pyralis
aacuuacgcugaguacuucga

SEQ ID NO: 25 - DNA, 21 bp, Mus musculus
acauugucacugcuaucuatt

SEQ ID NO: 26 - DNA, 21 bp, Mus musculus
uagauagcagugacaaugutt

SEQ ID NO: 27 - DNA, 21 bp, Mus musculus
ccacaaaugaagacuuauatt

SEQ ID NO: 28 - DNA, 19 bp, Mus musculus
gatcggagctgcaagattc

SEQ ID NO: 29 - DNA, 21 bp, Mus musculus
uauaagucuucauuuguggtt

SEQ ID NO: 30 - DNA, 19 bp, Mus musculus
tcgggatgtcacacaatgc

SEQ ID NO: 31 - DNA, 19 bp, Mus musculus
ttgtcggtgcaatagttcc

SEQ ID NO: 32 - RNA, 19 nt, Homo sapiens
acuccaguugccagugauc

SEQ ID NO: 33 - RNA, 19 nt, Homo sapiens
cgagacgccugguuacuau

SEQ ID NO: 34 - RNA, 19 nt, Homo sapiens
gacggcaguguauuagaga

SEQ ID NO: 35 - RNA, 19 nt, Homo sapiens
guguaacugguagcaagag

(19) United States
(12) Patent Application Publication
(10) Pub. No.: US 2006/0063731 A1
(43) Pub. Date: Mar. 23, 2006

(54) In vivo inhibition of hepatitis B virus

(76) Inventors: David L. Lewis, Madison, WI (US); Jon A. Wolff, Madison, WI (US); Hans Herweijer, Brooklyn, WI (US); James E. Hagstrom, Middleton, WI (US); Aaron G. Loomis, Prairie du Sac, WI (US)

Correspondence Address: MIRUS CORPORATION, 505 SOUTH ROSA RD, MADISON, WI, 53719, US

(21) Appl. No.: 11/234,947

(22) Filed: Sep. 26, 2005

Related U.S. Application Data
(63) Continuation-in-part of application No. 10/874,528, filed on Jun. 23, 2004.
(60) Provisional application No. 60/613,845, filed on Sep. 28, 2004.
(60) Provisional application No. 60/613,844, filed on Sep. 28, 2004.
(60) Provisional application No. 60/482,195, filed on Jun. 24, 2003.
(60) Provisional application No. 60/503,834, filed on Sep. 17, 2003.
(60) Provisional application No. 60/514,850, filed on Oct. 27, 2003.
(60) Provisional application No. 60/515,532, filed on Oct. 29, 2003.
(60) Provisional application No. 60/547,718, filed on Feb. 25, 2004.

(51) Int. Cl. A61K 48/00

(52) U.S. Cl. 514/44

(57) ABSTRACT

A process is provided to deliver polynucleotide-based gene expression inhibitors to cells in a mammal in vivo for the purpose of inhibiting gene expression in the cells. Inhibition is sequence-specific and relies on sequence similarity to of the polynucleotide-based gene expression inhibitor and the target nucleic acid molecule. Delivery of the polynucleotide-based gene expression inhibitor can enhance the efficacy of co-delivered small molecule drugs.

### CROSS-REFERENCE TO RELATED INVENTIONS

This application is a continuation-in-part of application Ser. No. 10/874,528, filed Jun. 23, 2004, pending, and claims the benefit of U.S. Provisional Applications 60/613,845, filed Sep. 28, 2004 and 60/613,844, filed Sep. 28, 2004. Application Ser. No. 10/874,528 claims the benefit of U.S. Provisional Applications 60/482,195, filed Jun. 24, 2003, 60/503,834 filed Sep. 17, 2003, 60/514,850 filed Oct. 27, 2003, 60/515,532 filed Oct. 29, 2003, and 60/547,718, filed Feb. 25, 2004. Application Ser. No. 10/874,528 is incorporated herein by reference.

### BACKGROUND OF THE INVENTION

The delivery of genetic material as a therapeutic, gene therapy, promises to be a revolutionary advance in the treatment of disease. Although, the initial motivation for gene therapy was the treatment of genetic disorders, it is becoming increasingly apparent that gene therapy will be useful for the treatment of a broad range of acquired diseases such as cancer, infectious disorders (AIDS), heart disease, arthritis, and neurodegenerative disorders (Parkinson's and Alzheimer's). Not only can functional genes be delivered to repair a genetic deficiency, but nucleic acid can also be delivered to inhibit gene expression to provide a therapeutic effect. Inhibition of gene expression can be affected by antisense polynucleotides, siRNA mediated RNA interference and ribozymes. Transfer methods currently being explored for delivering nucleic acids to cell in vivo include viral vectors and physical-chemical, or non-viral, methods.

RNA interference (RNAi) describes the phenomenon whereby the presence of double-stranded RNA (dsRNA) of sequence that is identical or highly similar to a target gene results in the degradation of messenger RNA (mRNA) transcribed from that target gene (Sharp 2001). RNAi is a natural cellular process that has recently been harnessed for a rapidly growing number of scientific, biotechnological, and therapeutic applications. In eukaryotic cells some long, double stranded RNA (dsRNA) molecules are processed into short fragments of 21-25 base pairs with two or three overhanging 3′ nucleotides on both ends. These fragments are able to initiate the sequence-specific cleavage, and thus inactivation, of single stranded RNA (ssRNA) molecules containing a homologous sequence motif (typically messenger RNA, mRNA). More recently, it has been shown that <30 bp siRNAs (small interfering RNAs) and miRNAs (microRNAs), delivered to a cell, induce RNAi in mammalian cells in culture and in vivo (Tuschl et al. 1999; Elbashir et al. 2001). Gene silencing can also be initiated in mammalian cells by transfection with an expression vector producing the RNAi nucleic acids using the cells' own transcription machinery.

There are two major approaches to initiate RNAi-mediated silencing in mammalian cells. First, synthetic siRNA or miRNA duplexes (typically between 19-30 base pairs in length) can be designed and generated against any known expressed gene sequence using guidelines known in the art. Second, expression cassettes that will generate RNAi nucleic acids within the cell can be delivered to the cell. Expression cassettes can take advantage of RNA Polymerase III (Pol-III) promoters or RNA Polymerase II (Pol-II) promoters. The two basic types of siRNA expression constructs code either for a hairpin RNA containing both the sense and the antisense sequence, separated by a loop region, or two separate promoters driving the transcription of the sense and antisense RNA strand separately.

The ability to specifically inhibit expression of a target gene by RNAi has obvious benefits. For example, RNAi could be used to generate animals that mimic true genetic “knockout” animals to study gene function. In addition, many diseases arise from the abnormal expression of a particular gene or group of genes. For example, genes contributing to a cancerous state or causing dominant genetic diseases such as myotonic dystrophy could be inhibited. In addition, viral genes may be inhibited. Inhibiting such genes as cyclooxygenase or cytokines could also treat inflammatory diseases such as arthritis. The ability to safely delivery siRNA to mammalian cells in vivo has profound potential for the treatment of infections and diseases as well as drug discovery and target validation.

Chronic hepatitis B is caused by an enveloped virus, commonly known as the hepatitis B virus or HBV. HBV is transmitted via infected blood or other body fluids, especially saliva and semen, during delivery, sexual activity, or sharing of needles contaminated by infected blood. Transmission is also possible via tattooing, ear or body piercing, and acupuncture; the virus is also stable on razors, toothbrushes, baby bottles, eating utensils, and some hospital equipment such as respirators, scopes and instruments. Individuals can be “carriers” and transmit the infection to others without ever having experienced symptoms of the disease. The average incubation period is 60 to 90 days, with a range of 45 to 180. The number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of disease severity include: (1) The size of the dose to which the person was exposed; (2) the person's age with younger patients experiencing a milder form of the disease; (3) the status of the immune system with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and fatigue. Jaundice is not experienced in all cases, however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement can occur accompanied by right upper-quadrant pain. The acute phase of the disease can be accompanied by severe depression, meningitis, Guillain-Barr syndrome, myelitis, encephalitis, agranulocytosis, and/or thrombocytopenia.

Hepatitis B is generally self-limiting and resolves in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life become carriers.

Hepatitis B virus (HBV) infects over 300 million people worldwide (Imperial, 1999, Gastroenterol. Hepatol., 14 (suppl), S1-5). In the United States approximately 1.25 million individuals are chronic carriers of HBV as evidenced by measurable hepatitis B virus surface antigen, HBsAg, in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation has been the only treatment option for patients with end-stage liver disease from HBV.

The natural progression of chronic HBV infection over a 10 to 20 year period leads to cirrhosis in 20 to 50% of patients and progression of HBV infection to hepatocellular carcinoma has been well documented.

Survival for patients diagnosed with hepatocellular carcinoma is only 0.9 to 12.8 months from initial diagnosis. Treatment of hepatocellular carcinoma with chemotherapeutic agents has not proven effective and only 10% of patients benefit from surgery due to extensive tumor invasion of the liver. Currently the only viable treatment alternative to surgery is liver transplantation.

Upon progression to cirrhosis, patients with chronic HBV or HCV infection present with clinical features, which are common to clinical cirrhosis regardless of the initial cause. These clinical features can include: bleeding esophageal varices, ascites, jaundice, and encephalopathy. In the early stages of cirrhosis, classified as compensated, the patient's liver is still able to detoxify metabolites in the bloodstream. Most patients with compensated liver disease are asymptomatic and the minority with symptoms report only minor symptoms such as dyspepsia and weakness. In the later stages of cirrhosis, decompensated, the ability to detoxify metabolites in the bloodstream is diminished.

A study by D'Amico (1986) indicated that the five year survival rate for all cirrhosis patients was only 40%. The six year survival rate for the patients who initially had compensated cirrhosis was 54% while the six year survival rate for patients who initially presented with decompensated disease was only 21%. The major causes of death for the patients in the D'Amico study were liver failure in 49%; hepatocellular carcinoma in 22%; and, bleeding in 13%.

Hepatitis B virus is a double-stranded circular DNA virus. It is a member of the Hepadnaviridae family. The virus is 42 nm in diameter, consisting of a central core that contains a core antigen (HBcAg) surrounded by an envelope containing a surface protein/surface antigen (HBsAg). It also contains an e antigen (HBeAg) that, along with HBcAg and HBsAg, is helpful in identifying this disease. In HBV virions, the genome is found in an incomplete double-stranded form. HBV uses a reverse transcriptase to transcribe a positive-sense full length RNA version of its genome back into DNA. This reverse transcriptase also contains DNA polymerase activity, and thus, begins replicating the newly synthesized minus-sense DNA strand.

Current therapeutic goals of treatment are three-fold: to eliminate infectivity and transmission of HBV to others, to arrest the progression of liver disease and improve the clinical prognosis, and to prevent the development of hepatocellular carcinoma (HCC).

Interferon alpha is the most common therapeutic for HBV. A complete response (HBV DNA negative HBeAg negative) occurs in approximately 25% of patients. The FDA has also recently approved Lamivudine (3TC.RTM.) as a therapeutic. There is a risk of reactivation of the hepatitis B virus even after a successful response, this occurs in around 5% of responders and normally occurs within 1 year.

Lamivudine (3TC™) is a nucleoside analogue, which is a very potent and specific inhibitor of HBV DNA synthesis. Unlike treatment with interferon, treatment with 3TC™ does not eliminate HBV from the patient. Rather, viral replication is controlled and chronic administration results in improvements in liver histology in over 50% of patients. Therefore, cessation of therapy results in reactivation of HBV replication in most patients. In addition, recent reports have documented 3TC.RTM. resistance in approximately 30% of patients. Thus, a need exists for effective treatment of this disease.

The intravascular delivery of nucleic acid has been shown to be highly effective for gene transfer into tissue in vivo (U.S. application Ser. No. 09/330,909, U.S. Pat. No. 6,627,616). Non-viral vectors are inherently safer than viral vectors, have a reduced immune response induction and have significantly lower cost of production. Furthermore, a much lower risk of transforming activity is associated with non-viral polynucleotides than with viruses.

### SUMMARY OF THE INVENTION

In a preferred embodiment we describe processes for delivering a RNA function inhibitor (hereafter referred to as “inhibitor”) to an animal cell. We also describe compositions that facilitate delivery of an inhibitor to an animal cell. Delivery of the inhibitor results in inhibition of target gene expression by causing degradation of inhibition of function of RNA. Inhibitors are selected from the group comprising siRNA, dsRNA, antisense nucleic acid, ribozymes, RNA polymerase III transcribed DNAs, microRNA, and the like. A preferred inhibitor is siRNA.

In a preferred embodiment, we describe an in vivo process for delivery of an inhibitor to a cell of a mammal for the purposes of inhibition of gene expression (RNA function) comprising: making an inhibitor, injecting the inhibitor into a vessel, and delivering the inhibitor to a cell within a tissue thereby inhibiting expression of a target gene in the cell. Permeability of the vessel to the inhibitor may comprise increasing the pressure within the vessel by rapidly injecting a large volume of fluid into the vessel and, for some target tissues, blocking the flow of fluid into and/or out of the target tissue. This increased pressure is controlled by altering the injection volume, altering the rate of volume insertion, and by constricting the flow of blood into or out of the tissue during the procedure. The volume consists of an inhibitor in a solution wherein the solution may contain a compound or compounds which may or may not complex with the inhibitor and aid in delivery.

In a preferred embodiment, a process is described for increasing the transit of the inhibitor out of a vessel and into the cells of the surrounding tissue, comprising rapidly injecting a large volume into a vessel supplying the target tissue, thus forcing fluid out of the vasculature into the extravascular space. This process is accomplished by forcing a large volume containing the inhibitor into a vessel and, for some tissues, constricting the flow of fluid into and/or out of an area. Optionally, a molecule that increases the permeability of a vessel may be included in the volume. The target tissue comprises the cells supplied by the vessel. For injection into arteries, the target tissue is the cells that the arteries supply with blood. For injection into veins, the target tissue is the cells from which the vein drains blood.

In a preferred embodiment, we describe a process for inhibiting gene expression in an animal cell comprising: delivering of one or more small RNAi molecules to the cell. The RNAi molecules comprise a sequence that is identical, nearly identical, or complementary to the same, different, or overlapping segments of a target gene sequence(s). The RNAi molecules may be formed outside the cell and then delivered to the cell. Alternatively, the RNAi molecules may be transcribed within the cell from of a nucleic acid that is delivered to the cell.

The RNAi molecules may be delivered to cells in vivo, ex vivo, in situ, or in vitro. The cell can be an animal cell that is maintained in tissue culture such as cell lines that are immortalized or transformed. The cell can be a primary or secondary cell which means that the cell has been maintained in culture for a relatively short time after being obtained from an animal. The cell can also be a mammalian cell that is within a tissue in situ or in vivo meaning that the cell has not been removed from the tissue or the animal.

In a preferred embodiment the RNAi molecules may be modified by association or attachment of a functional group. The functional group can be, but is not limited to, a transfection reagent, targeting signal or a label or other group that facilitates delivery of the inhibitor.

In a preferred embodiment, a combination of two or more inhibitors are delivered together or sequentially to enhance inhibition of target gene expression. The inhibitors comprise sequence that is identical, nearly identical, or complementary to the same, different, or overlapping segments of the target gene sequence(s). For instance, a preferred combination comprises one inhibitor that is a small RNAi molecules and another inhibitor that is an antisense polynucleotide. A preferred antisense polynucleotide is a morpholino or a 2′-O-methyl oligonucleotide. The inhibitors may be delivered to cells in vivo, ex vivo, in situ, or in vitro.

In a preferred embodiment, we describe a process for the simultaneous or coordinated delivery of an small RNAi molecules together with a small molecule drug to a cell or tissue, i.e. combination therapy. The RNAi molecules is delivered to the cell or tissue to exert an effect on the levels of a protein, such as an enzyme, in the cell or tissue. The RNAi-induced reduction in the amount the protein can enhance or alter the effect of a small molecule drug. In a preferred embodiment, a lower dose of the small molecule is required to generate a specific cellular outcome when combined with RNAi molecule delivery. By using RNAi molecule to reduce the amount of a target protein, the dose of drug required to inhibit an endogenous cellular protein is lowered or its efficacy is increased. The drug and the RNAi molecule may both affect the same gene/gene product. Alternatively, the RNAi molecule and drug may be chosen to work cooperatively through inhibition of different genes.

In a preferred embodiment, an inhibitor may be delivered to a cell in a mammal for the purposes of inhibiting a target gene to provide a therapeutic effect. The target gene is selected from the group that comprises: dysfunctional endogenous genes and viral or other infectious agent genes. Dysfunctional endogenous genes include dominant genes which cause disease and cancer genes. A preferred viral gene is a hepatitis B virus gene. In one embodiment, the inhibitor is delivered to an HBV infected patient. In another embodiment, the patient is one who does not respond to treatment with interferon, interferon-related therapeutics, or 3TC™ (Lamivudine). Delivery of the inhibitor may be combined with treatment with other drugs. In a preferred embodiment, the likelihood of success for any given inhibitor may be tested in animal hepatitis models such as described in U.S. patent Publication 20030140362.

In a preferred embodiment, an inhibitor is delivered to a mammalian cell in vivo for the treatment of a disease or infection. The inhibitor reduces expression of a viral or bacterial gene. The inhibitor may reduce or block microbe production, virulence, or both. Delivery of the inhibitor may delay progression of disease until endogenous immune protection can be acquired. In a preferred embodiment, combinations of effective inhibitors or combinations of inhibitor and small molecule drugs targeted to the same or different viral genes or classes of genes (e.g., transcription, replication, virulence, etc) are delivered to an infected mammalian cell in vivo. Alternatively, instead of inhibiting an infectious agent gene, the inhibitor may decrease expression of an endogenous host gene to reduce virulence of the pathogen. The inhibitor may be delivered to a cell in a mammal to reduce expression of a cellular receptor.

In a preferred embodiment, an inhibitor is delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy, where immune reaction often greatly limits transgene expression, organ transplantation, and autoimmune disorders.

In a preferred embodiment, an inhibitor is delivered to a mammalian cell for the purpose of facilitating pharmaceutical drug discovery or target validation. The mammalian cell may be in vitro or in vivo. Specific inhibition of a target gene can aid in determining whether an inhibition of a protein or gene has a significant phenotypic effect. Specific inhibition of a target gene can also be used to study the target gene's effect on the cell.

Further objects, features, and advantages of the invention will be apparent from the following detailed description when taken in conjunction with the accompanying drawings.

### BRIEF DESCRIPTION OF FIGURES

FIG. 1. siRNA is efficiently delivered to multiple tissue types in mice in vivo and the delivered siRNA is highly effective for inhibiting target gene expression in all organs tested.

FIG. 2. Intravascular delivery of siRNA inhibits EGFP expression in the liver of transgenic mice. EGFP (green), phalloidin (red). 10 week old mice (strain C57BL/6-TgN (ACTbEGFP) 10sb) expressing EGFP were injected with 50 μg siRNA (mice #1 and 2), 50 μg control siRNA (mice #3 and 4) or were not injected (mouse #5). Livers were harvested 30 h post-injection, sectioned, fixed, and counterstained with Alexa 568 phalloidin in order to visualize cell outlines. Images were acquired using a Zeiss Axioplan fluorescence microscope outfitted with a Zeiss AxioCam digital camera.

FIG. 3. Graph illustrating reduction in PPAR levels following delivery of PPAR-siRNA expression cassettes in vivo.

FIG. 4A-4B. A. Graph illustrating levels of HMG-CoA reductase mRNA in mice treated with 50 mg/kg atorvastatin. B. Graph illustrating prevention of atorvastatin-induced upregulation of HMGCR levels in vitro by co-delivery of HMGCR siRNA.

FIG. 5. Relative levels of PPARα mRNA in groups of mice injected with siRNAs. mRNA levels are shown relative to total input RNA. Black bar=Experimental group; Grey bars=control group.

FIG. 6A-6C. A. Graph illustrating effect of statin treatment on LDLR mRNA in primary hepatocytes. B. Graph illustrating relative levels of LDLR mRNA in hepatocytes treated with statins and siRNAs. Dark bars=HMGCR siRNA-treated cells; Light bars=GL3-treated cells. C. Graph illustrating lower doses of atorvastatin necessary to get comparable statin/no statin ratios in cells treated with HMGCR siRNAs.

FIG. 7. Graph Illustrating in vivo inhibition of Hepatitis B surface antigen (HBsAg) following delivery of HepB-sAG specific siRNA via intravascular injection.

FIG. 8. Graph Illustrating in vivo inhibition of Hepatitis B surface antigen (HBsAg) following delivery of HepB-sAG specific siRNA via intravascular injection.

FIG. 9. Confocal microscope images illustrating siRNA mediated silencing the Ki-67 expression in HeLa cells. A. Untreated control cells. B. Non-specific control siRNA. C.-D. Ki-67 siRNA MK167 #3. Ki-67 protein localization=upper left panels; Phalloidin stained actin=upper right panels; To-Pro3 stained DNA=lower left panels; composite images=lower right panels. Arrows point at cells in various stages of mitosis.

FIG. 10. Chart illustrating dose-dependent silencing of Ki-67 by MK167 #3 siRNA in HeLa cells.

FIG. 11. Confocal microscope images of HeLa cells: A-B. untreated; C. transfected twice with 50 nM negative control siRNA; or, D. transfected twice with 50 nM MK167 #3 siRNA. Ki-67 detection=upper left panels; Fluorescein-12-dUTP-labeled nucleotides incorporated into fragmented DNA in nuclei of apoptotic (B, C, D) and DNAase-treated positive control cells (A)=upper right panels. To-Pro3 stained DNA=lower left panels; composite images=lower right panels.

### DETAILED DESCRIPTION

We have found that an intravascular route of administration allows a polynucleotide-based expression inhibitor (inhibitor) to be delivered to a mammalian cell in a more even distribution than is accomplished with direct parenchymal injections. The efficiency of inhibitor delivery may be increased by increasing the permeability of the tissue's blood vessel. Permeability is increased by increasing the intravascular hydrostatic pressure (above, for example, the resting diastolic blood pressure in a blood vessel), delivering the injection fluid rapidly (injecting the injection fluid rapidly), using a large injection volume, and/or increasing permeability of the vessel wall.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. RNAi molecules are polynucleotides or polynucleotide analogs that, when delivered to a cell, inhibit RNA function through RNA interference. Small RNAi molecules include RNA molecules less that about 50 nucleotides in length and include siRNA and miRNA. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

A delivered inhibitor can stay within the cytoplasm or nucleus. The inhibitor can be delivered to a cell to inhibit expression of an endogenous or exogenous nucleotide sequence or to affect a specific physiological characteristic not naturally associated with the cell.

An inhibitor can be delivered to a cell in order to produce a cellular change that is therapeutic. Entry into the cell is required for the inhibitor to block the production of a protein or to decrease the amount of a target RNA. Diseases, such as autosomal dominant muscular dystrophies, which are caused by dominant mutant genes, are examples of candidates for treatment with therapeutic inhibitors such as siRNA. Delivery of the inhibitor would block production of the dominant protein without affecting the normal protein thereby lessening the disease.

Inhibitors may also be delivered to a cell in a mammal to inhibit a viral infection. The inhibitor can by delivered to reduces expression of a viral or bacterial gene. The inhibitor may reduce or block microbe production, virulence, or both. Delivery of the inhibitor may delay progression of disease until endogenous immune protection can be acquired. Viral genes involved in transcription, replication, virion assembly, immature viral membrane formation, extracellular enveloped virus formation, early genes, intermediate genes, late genes, and virulence genes may be targeted. Bacterial genes involved in transcription, replication, virulence, cell growth, pathogenicity, etc. may be targeted. Combinations of effective inhibitors targeted to the same or different viral genes or classes of genes (e.g., transcription, replication, virulence, etc) can be delivered to an infected mammalian cell in vivo.

An inhibitor may be delivered to decrease expression of an endogenous host gene to reduce virulence of the pathogen. The inhibitor may be delivered to a cell in a mammal to reduce expression of a cellular receptor. For example, the lethality of Anthrax is primarily mediated by a secreted tripartite toxin which requires the mammalian anthrax toxin receptor (ATR) for cellular entry. Reducing expression of ATR may decrease Anthrax toxicity. Receptors to which pathogens bind may also be targeted.

An inhibitor may also be delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy, where immune reaction often greatly limits transgene expression, organ transplantation, and autoimmune disorders.

Any gene, known in the art, whose expression is known to contribute to viral or bacterial infection or to pathogenicity or toxicity may be a target for polynucleotide-based gene expression inhibitors.

We demonstrate that delivery of siRNA and antisense inhibitors to cells of post-embryonic mice and rats interferes with specific gene expression in those cells. The inhibition is gene specific and does not cause general translational arrest. Thus RNAi can be effective in post-embryonic mammalian cells in vivo.

Many disease treatments aim to inhibit the activity of a well-defined protein to give a therapeutic effect. Such effects are realized only when the levels of active target protein drop below a certain threshold. SiRNA may be used to reduce the amount of target protein to be inhibited by small molecule drugs. This reduction in protein levels results in a lower dosage of the small molecule drug be necessary to gain a clinical outcome, perhaps leading to significantly lower recommended doses and reduced side effects. This strategy may help lower the hurdles to successful treatments for a variety of diseases. In addition, it may facilitate drug discovery and research by providing a method of sensitizing cells to the action of a small molecule targeting a particular gene product.

Combination therapy is defined as the simultaneous administration of multiple treatments to treat a single pathogenic or disease state. This strategy has been used successfully to treat a variety of diseases. For example, chemotherapy and radiation remain a common treatment of nearly all cancers. Furthermore, many of the newer anti-cancer drugs are measured for efficacy in combination with traditional therapies like chemotherapy and radiation. In addition, HIV combination therapy and its cocktail of protease inhibitors and reverse transcriptase inhibitors has returned a sort of normalcy to the lives of many AIDS patients.

The term nucleic acid, or polynucleotide, is a term of art that refers to a string of at least two nucleotides. Nucleotides are the monomeric units of nucleic acid polymers. Polynucleotides with less than 120 monomeric units are often called oligonucleotides. Natural nucleic acids have a deoxyribose- or ribose-phosphate backbone while artificial polynucleotides are polymerized in vitro and contain the same or similar bases but may contain other types of backbones. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups on the base such as, but not limited to, amines, alcohols, thiols, carboxylates, and alkylhalides. The term base encompasses any of the known base analogs of DNA and RNA. The term includes deoxyribonucleic acid (DNA) and ribonucleic acid (RNA). DNA may be in form of cDNA, in vitro polymerized DNA, plasmid DNA, parts of a plasmid DNA, genetic material derived from a virus, linear DNA, chromosomal DNA, an oligonucleotide, antisense DNA, or derivatives of these groups. RNA may be in the form of tRNA (transfer RNA), snRNA (small nuclear RNA), rRNA (ribosomal RNA), mRNA (messenger RNA), antisense RNA, siRNA (small interfering RNA), dsRNA (double stranded RNA), RNAi, ribozymes, in vitro polymerized RNA, or derivatives of these groups.

The term deliver means that the inhibitor becomes associated with the cell thereby altering the properties of the cell by inhibiting function of an RNA. The sites of action for the inhibitors of the invention are the cytoplasm and nucleus. Other terms sometimes used interchangeably with deliver include transfect, transfer, or transform. In vivo delivery of an inhibitor means to transfer the inhibitor from a container outside a mammal to within the outer cell membrane of a cell in the mammal. The inhibitor can interfere with RNA function in either the nucleus or cytoplasm.

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

For example, in a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the inhibitor or inhibitor complex into the portal vein or bile duct of a mammal.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

In a preferred embodiment, the permeability of the vessel is increased. Efficiency of inhibitor delivery is increased by increasing the permeability of a vessel within the target tissue. Permeability is defined here as the propensity for macromolecules such as an inhibitor to exit the vessel and enter extravascular space. One measure of permeability is the rate at which macromolecules move out of the vessel. Another measure of permeability is the lack of force that resists the movement of inhibitors being delivered to leave the intravascular space.

Rapid injection may be combined with obstructing the outflow to increase permeability. To obstruct, in this specification, is to block or inhibit inflow or outflow of fluid through a vessel. For example, an afferent vessel supplying an organ is rapidly injected and the efferent vessel draining the tissue is ligated transiently. The efferent vessel (also called the venous outflow or tract) draining outflow from the tissue is also partially or totally clamped for a period of time sufficient to allow delivery of a polynucleotide. In the reverse, an efferent is injected and an afferent vessel is occluded.

In another preferred embodiment, the pressure of a vessel is increased by increasing the osmotic pressure within the vessel. Typically, hypertonic solutions containing salts such as NaCl, sugars or polyols such as mannitol are used. Hypertonic means that the osmolarity of the injection solution is greater than physiological osmolarity. Isotonic means that the osmolarity of the injection solution is the same as the physiological osmolarity (the tonicity or osmotic pressure of the solution is similar to that of blood). Hypertonic solutions have increased tonicity and osmotic pressure relative to the osmotic pressure of blood and cause cells to shrink.

In another preferred embodiment, the permeability of a vessel can be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Biologically-active molecules include vascular permeability factor (VPF) which is also known as vascular endothelial growth factor (VEGF). Another type of biologically-active molecule can increase permeability by changing the extracellular connective material. For example, an enzyme could digest the extracellular material and increase the number and size of the holes of the connective material.

In a preferred embodiment, an inhibitor or inhibitor-containing complex is injected into a vessel in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volumes in terms of ml/body weight can be 0.03 ml/g to 0.1 ml/g or greater.

The injection volume can also be related to the target tissue. For example, delivery of a non-viral vector with an inhibitor to a limb can be aided by injecting a volume greater than 5 ml per rat limb or greater than 70 ml for a primate. The injection volumes in terms of ml/limb muscle are usually within the range of 0.6 to 1.8 ml/g of muscle but can be greater. In another example, delivery of an inhibitor or inhibitor complex to liver in mice can be aided by injecting the inhibitor in an injection volume from 0.6 to 1.8 ml/g of liver or greater. In another example delivering an inhibitor to a limb of a primate (rhesus monkey), the inhibitor or complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel into which the volume is injected, and the size of the animal. In one embodiment the total injection volume (1-3 ml) can be injected from 15 to 5 seconds into the vascular system of mice. In another embodiment the total injection volume (6-35 ml) can be injected into the vascular system of rats from 20 to 7 seconds. In another embodiment the total injection volume (80-200 ml) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than 0.2 ml per gram (target tissue weight) per second are used for gene delivery to target organs. In another embodiment injection rates of less than 0.06 ml per gram (target tissue weight) per second are used for gene delivery into limb muscle and other muscles of primates.

Polymers have been used in research for the delivery of nucleic acids to cells. One of the several methods of nucleic acid delivery to the cells is the use of nucleic acid/polycation complexes. It has been shown that cationic proteins, like histones and protamines, or synthetic polymers, like polylysine, polyarginine, polyomithine, DEAE dextran, polybrene, and polyethylenimine, but not small polycations like spermine may be effective intracellular DNA delivery agents. Multivalent cations with a charge of three or higher have been shown to condense nucleic acid when 90% or more of the charges along the sugar-phosphate backbone are neutralized. The volume which one polynucleotide molecule occupies in a complex with polycations is lower than the volume of a free polynucleotide molecule. Polycations also provide attachment of polynucleotide to a cell surface. The polymer forms a cross-bridge between the polyanionic nucleic acid and the polyanionic surface of the cell. As a result, the mechanism of nucleic acid translocation to the intracellular space might be non-specific adsorptive endocytosis. Furthermore, polycations provide a convenient linker for attaching specific ligands to the complex. The nucleic acid/polycation complexes could then be targeted to specific cell types. Complex formation also protects against nucleic acid degradation by nucleases present in serum as well as in endosomes and lysosomes. Protection from degradation in endosomes/lysosomes is enhanced by preventing organelle acidification. Disruption of endosomal/lysosomal function may also be accomplished by linking endosomal or membrane disruptive agents to the polycation or complex.

A DNA-binding protein is a protein that associates with nucleic acid under conditions described in this application and forms a complex with nucleic acid with a high binding constant. The DNA-binding protein can be used in an effective amount in its natural form or a modified form for this process. An “effective amount” of the polycation is an amount that will allow delivery of the inhibitor to occur.

A non-viral vector is defined as a vector that is not assembled within an eukaryotic cell including non-viral inhibitor/polymer complexes, inhibitor with transfection enhancing compounds and inhibitor+ amphipathic compounds.

A molecule is modified, to form a modification through a process called modification, by a second molecule if the two become bonded through a covalent bond. That is, the two molecules form a covalent bond between an atom from one molecule and an atom from the second molecule resulting in the formation of a new single molecule. A chemical covalent bond is an interaction, bond, between two atoms in which there is a sharing of electron density. Modification also means an interaction between two molecules through a noncovalent bond. For example crown ethers can form noncovalent bonds with certain amine groups.

Functional groups include cell targeting signals, nuclear localization signals, compounds that enhance release of contents from endosomes or other intracellular vesicles (releasing signals), and other compounds that alter the behavior or interactions (interaction modifiers) of the compound are complex to which they are attached. Polyethylene glycol and other hydrophilic polymers have provided protection of the pharmaceutical in the blood stream by preventing its interaction with blood components and to increase the circulatory time of the pharmaceutical by preventing opsonization, phagocytosis and uptake by the reticuloendothelial system.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asialoglycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be delivered to the cell for research purposes or to produce a change in a cell that can be therapeutic. The delivery of a polynucleotide for therapeutic purposes is commonly called gene therapy. Gene therapy is the purposeful delivery of genetic material to somatic cells for the purpose of treating disease or biomedical investigation. The delivery of a polynucleotide can lead to modification of the genetic material present in the target cell.

Transfection agent—A transfection reagent or delivery vehicle is a compound or compounds that bind(s) to or complex(es) with oligonucleotides and polynucleotides, and mediates their entry into cells. Examples of transfection reagents include, but are not limited to, cationic liposomes and lipids, polyamines, calcium phosphate precipitates, histone proteins, polyethylenimine, polylysine, and polyampholyte complexes. It has been shown that cationic proteins like histones and protamines, or synthetic polymers like polylysine, polyarginine, polyornithine, DEAE dextran, polybrene, and polyethylenimine may be effective intracellular delivery agents. Typically, the transfection reagent has a component with a net positive charge that binds to the oligonucleotide's or polynucleotide's negative charge.

Biologically active compound—A biologically active compound is a compound having the potential to react with biological components. More particularly, biologically active compounds utilized in this specification are designed to change the natural processes associated with a living cell. For purposes of this specification, a cellular natural process is a process that is associated with a cell before delivery of a biologically active compound. Biologically active compounds may be selected from the group comprising: pharmaceuticals, drugs, proteins, peptides, polypeptides, hormones, cytokines, antigens, viruses, oligonucleotides, and nucleic acids.

We have disclosed gene expression and/or inhibition achieved from reporter genes in specific tissues. Levels of a gene product, including reporter (marker) gene products, are measured which then indicate a reasonable expectation of similar amounts of gene expression by delivering other polynucleotides. Levels of treatment considered beneficial by a person having ordinary skill in the art differ from disease to disease, for example: Hemophilia A and B are caused by deficiencies of the X-linked clotting factors VIII and IX, respectively. Their clinical course is greatly influenced by the percentage of normal serum levels of factor VIII or IX: <2%, severe; 2-5%, moderate; and 5-30% mild. Thus, an increase from 1% to 2% of the normal level of circulating factor in severe patients can be considered beneficial. Levels greater than 6% prevent spontaneous bleeds but not those secondary to surgery or injury. A person having ordinary skill in the art of gene therapy would reasonably anticipate beneficial levels of expression of a gene specific for a disease based upon sufficient levels of marker gene results. In the hemophilia example, if marker genes were expressed to yield a protein at a level comparable in volume to 2% of the normal level of factor VIII, it can be reasonably expected that the gene coding for factor VIII would also be expressed at similar levels. Thus, reporter or marker genes such as the genes for luciferase and β-galactosidase serve as useful paradigms for expression of intracellular proteins in general. Similarly, reporter or marker genes, such as the gene for secreted alkaline phosphatase (SEAP), serve as useful paradigms for secreted proteins in general.

### EXAMPLES

The following examples are intended to illustrate, but not limit, the present invention.

### Example 1

Inhibition of luciferase gene expression by siRNA in liver cells in vivo. Single-stranded, gene-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use.

The sense oligomer with identity to the luc+ gene has the sequence: 5′-rCrUrUrArCrGrC-rUrGrArGrUrArCrUrUrCrGrATT-3′ (SEQ ID 4), which corresponds to positions155-173 of the luc+ reading frame. The letter “r” preceding a nucleotide indicates that nucleotide is a ribonucleotide. The antisense oligomer with identity to the luc+ gene has the sequence: 5′-rUrCrGrArArGrUrArCrUrCrArGrCrGrUrArArGTT-3′ (SEQ ID 5), which corresponds to positions155-173 of the luc+ reading frame in the antisense direction. The letter “r” preceding a nucleotide indicates that nucleotide is a ribonucleotide. The annealed oligomers containing luc+ coding sequence are referred to as siRNA-luc+.

The sense oligomer with identity to the ColE1 replication origin of bacterial plasmids has the sequence: 5′-rGrCrGrArUrArArGrUrCrGrUrGrUrCrUrUrArCTT-3′ (SEQ ID 6). The letter “r” preceding a nucleotide indicates that nucleotide is a ribonucleotide. The antisense oligomer with identity to the ColE1 origin of bacterial plasmids has the sequence: 5′-rGrUrArArGrArCrArCrGrArCrUrUrArUrCrGrCTT-3′ (SEQ ID 7). The letter “r” preceding a nucleotide indicates that nucleotide is a ribonucleotide. The annealed oligomers containing ColE1 sequence are referred to as siRNA-ori.

Plasmid pMIR48 (10 μg), containing the luc+ coding region (Promega Corp.) and a chimeric intron downstream of the cytomegalovirus major immediate-early enhancer/promoter, was mixed with 0.5 or 5 μg siRNA-luc+, diluted in 1-3 ml Ringer's solution (147 mM NaCl, 4 mM KCl, 1.13 mM CaCl₂) and injected into the tail vein of ICR mice over 7-120 seconds. One day after injection, the livers were harvested and homogenized in lysis buffer (0.1% Triton X-100, 0.1 M K-phosphate, 1 mM DTT, pH 7.8). Insoluble material was cleared by centrifugation. 10 μl of the cellular extract or extract diluted 10× was analyzed for luciferase activity using the Enhanced Luciferase Assay kit (Mirus).

Co-injection of 10 μg pMIR48 and 0.5 μg siRNA-luc+ results in 69% inhibition of Luc+ activity as compared to injection of 10 μg pMIR48 alone. Co-injection of 5 μg siRNA-luc+ with 10 μg pMIR48 results in 93% inhibition of Luc+ activity.

### Example 2

Inhibition of Luciferase expression by siRNA is gene specific in liver in vivo. Two plasmids were injected simultaneously either with or without siRNA-luc+ as described in Example 1. The first plasmid, pGL3 control (Promega Corp, Madison, Wis.), contains the luc+ coding region and a chimeric intron under transcriptional control of the simian virus 40 enhancer and early promoter region. The second, pRL-SV40, contains the coding region for the Renilla reniformis luciferase under transcriptional control of the Simian virus 40 enhancer and early promoter region.

10 μg pGL3 control and 1 μg pRL-SV40 was injected as described in Example 1 with 0, 0.5 or 5.0 μg siRNA-luc+. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA-Luc+ control. siRNA-luc+ specifically inhibited the target Luc+ expression 73% at 0.5 μg co-injected siRNA-luc+ and 82% at 5.0 μg co-injected siRNA-luc+.

### Example 3

Inhibition of Luciferase expression by siRNA is gene specific and siRNA specific in liver in vivo. 10 μg pGL3 control and 1 μg pRL-SV40 were injected as described in Example 1 with either 5.0 μg siRNA-luc+ or 5.0 control siRNA-ori. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 93% compared to siRNA-ori indicating inhibition by siRNAs is sequence specific in this organ.

### Example 4

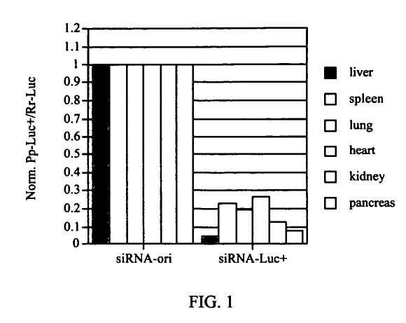

In vivo delivery of siRNA by increased-pressure intravascular injection results in strong inhibition of target gene expression in a variety of organs. 10 μg pGL3 Control and 1 μg pRL-SV40 were co-injected with 5 μg siRNA-Luc+ or 5 μg control siRNA (siRNA-ori) targeted to sequence in the plasmid backbone as in example 1. One day after injection, organs were harvested and homogenized and the extracts assayed for target firefly luciferase+ activity and control Renilla luciferase activity. Firefly luciferase+ activity was normalized to that Renilla luciferase activity in order to compensate for differences in transfection efficiency between animals. Results are shown in FIG. 1. Expression of firefly luciferase+ activity was strongly inhibited in liver (95% inhibition), spleen (77%), lung (81%), heart (74%), kidney (87%) and pancreas (92%), compared to animals injected with the control siRNA-ori. Animals injected with plasmid alone contained similar luciferase activities to those injected with the control siRNA-ori alone, indicating that the presence of siRNA alone does not significantly affect in vivo plasmid DNA transfection efficiencies (data not shown).

These results (FIG. 1) indicate effective delivery of siRNA to a number of different tissue types in vivo. Furthermore, the fact that expression of the control Renilla luciferase was not affected by the presence of siRNA suggests that siRNA is not inducing an interferon response. This is the first demonstration of the effectiveness of siRNA for inhibiting gene expression in post-embryonic mammalian tissues and demonstrates siRNA could be delivered to these organs to inhibit gene expression.

### Example 5

Inhibition of Luciferase expression by siRNA is gene specific and siRNA specific in liver after bile duct delivery in vivo. 10 μg pGL3 control and 1 μg pRL-SV40 with 5.0 μg siRNA-luc+ or 5.0 siRNA-ori were injected into the bile duct of mice. A total volume of 1 ml in Ringer's buffer was delivered at 6 ml/min. The inferior vena cava was clamped above and below the liver before injection and clamps were left on for two minutes after injection. One day after injection, the liver was harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 88% compared to the control siRNA-ori.

### Example 6

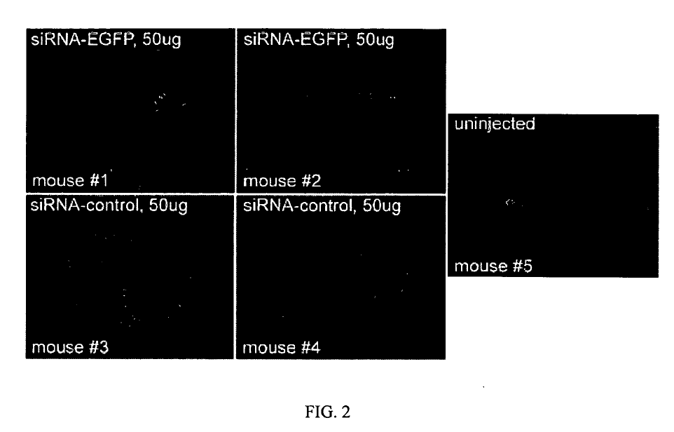

Inhibition of green fluorescent protein in transgenic mice using siRNA. The commercially available mouse strain C57BL/6-TgN(ACTbEGFP)10sb (The Jackson Laboratory) has been reported to express enhanced green fluorescent protein (EGFP) in all cell types except erythrocytes and hair. These mice were injected with siRNA targeted against EGFP (siRNA-EGFP) or a control siRNA (siRNA-control) using the increased pressure tail vein intravascular injection method described previously. 30 h post-injection, the animals were sacrificed and sections of the liver were prepared for fluorescence microscopy. Liver sections from animals injected with 50 μg siRNA-EGFP displayed a substantial decrease in the number of cells expressing EGFP compared to animals injected with siRNA-control or mock injected (FIG. 2). The data shown here demonstrate effective delivery of siRNA-EGFP to the liver. The delivered siRNA-EGFP then inhibited EGFP gene expression in the mice. We have therefore shown the ability of siRNA to inhibit the expression of an endogenous gene product in post-natal mammals.

### Example 7

Inhibition of endogenous mouse cytosolic alanine aminotransferase (ALT) expression after in vivo delivery of siRNA. Single-stranded, cytosolic alanine aminotransferase-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use. The sense oligomer with identity to the endogenous mouse and rat gene encoding cytosolic alanine aminotransferase has the sequence: 5′-rCrArCrUrCrArGrUrCrUrCrUrArArGrG-rGrCrUTT-3′ (SEQ ID 10), which corresponds to positions 928-946 of the cytosolic alanine aminotransferase reading frame in the sense direction. The letter “r” preceding a nucleotide indicates that nucleotide is a ribonucleotide. The sense oligomer with identity to the endogenous mouse and rat gene encoding cytosolic alanine aminotransferase has the sequence: 5′-rArGrCrCrCrUrUrArGrArGrArCrUrGrArGrUrGTT-3′ (SEQ ID 11), which corresponds to positions 928-946 of the cytosolic alanine aminotransferase reading frame in the antisense direction. The letter “r” preceding a nucleotide indicates that nucleotide is a ribonucleotide. The annealed oligomers containing cytosolic alanine aminotransferase coding sequence are referred to as siRNA-ALT

Mice were injected into the tail vein over 7-120 seconds with 40 μg siRNA-ALT diluted in 1-3 ml Ringer's solution (147 mM NaCl, 4 mM KCl, 1.13 mM CaCl₂). Control mice were injected with Ringer's solution without siRNA. Two days after injection, the livers were harvested and homogenized in 0.25 M sucrose. ALT activity was assayed using the Sigma diagnostics INFINITY ALT reagent according to the manufacturers instructions. Total protein was determined using the BioRad Protein Assay. Mice injected with 40 μg siRNA-ALT had an average decrease in ALT specific activity of 32% compared to mice injected with Ringer's solution alone.

### Example 8

Inhibition of Luciferase expression by delivery of antisense morpholino and siRNA simultaneously to liver in vivo. Morpholino antisense molecule and siRNAs used in this example were as follows:

DL94 morpholino (GeneTools Philomath, Oreg.), 5′-TTATGTTTTTGGCGTCTTCCATGGT-3′ (SEQ ID 1; Luc+ −3 to +22 of pGL3 Control Vector), was designed to base pair to the region surrounding the Luc+ start codon in order to inhibit translation of mRNA. Sequence of the start codon in the antisense orientation is underlined.

Standard control morpholino, 5′-CCTCTTACCTCAGTTACAATTTATA-3′ (SEQ ID 3), contains no significant sequence identity to Luc+ sequence or other sequences in pGL3 Control Vector

GL3 siRNA-Luc+: SEQ ID 4 and SEQ ID 5.

DL88:DL88C siRNA (targets EGFP 477-495, nt765-783):

(SEQ ID 12)

5′-rGrArArCrGrGrCrArUrCrArArGrGrUrGrArArCdTdT-3′

(SEQ ID 13)

5′-rGrUrUrCrArCrCrUrUrGrArUrCrCrCrGrUrUrCdTdT-3′

Two plasmid DNAs±siRNA and ±antisense morpholino in 1-3 ml Ringer's solution (147 mM NaCl, 4 mM KCl, 1.13 mM CaCl₂) were injected, in 7-120 seconds, into the tail vein of mice. The plasmids were pGL3 control, containing the luc+ coding region under transcriptional control of the simian virus 40 enhancer and early promoter region, and pRL-SV40, containing the coding region for the Renilla reniformis luciferase under transcriptional control of the Simian virus 40 enhancer and early promoter region. 2 μg pGL3 control and 0.2 μg pRL-SV40 were injected with or without 5.0 μg siRNA and with or without 50 μg DL94 morpholino. One day after injection, the livers were harvested and homogenized in lysis buffer (0.1% Triton X-100, 0.1M K-phosphate, 1 mM DTT, pH 7.8). Insoluble material were cleared by centrifugation. The homogenate was diluted 10-fold in lysis buffer and 5 μl was assayed for Luc+ and Renilla luciferase activities using the Dual Luciferase Reporter Assay System (Promega Corp.). Ratios of Luc+ to Renilla Luc were normalized to the 0 μg siRNA-Luc+ control.

TABLE 3
Inhibition of luciferase expression from pGL3 control
plasmid in mouse liver after delivery of 50 μg
antisense morpholino, 5 μg siRNA or both.

| | | percent inhibition of |
|---|---|---|
| Antisense morpholino | siRNA | luciferase expression |
| — | — | 0 |
| Standard | DL88:DL88C | 0 |
| DL94 | DL88:DL88C | 85.4 ± 2.7 |
| Standard | GL3 siRNA-Luc+ | 92.0 ± 1.9 |
| DL94 | GL3 siRNA-Luc+ | 98.6 ± 0.5 |

These experiments demonstrate the near complete inhibition of gene expression in vivo when antisense morpholino is delivered together with siRNA. This level if inhibition was greater than that for either morpholino of siRNA individually.

### Example 9

In vivo delivery of siRNA to mouse liver cells using TransIT™ In Vivo. 10 μg pGL3 control and 1 μg pRL-SV40 were complexed with 11 μl TransIT™ In Vivo in 2.5 ml total volume according the manufacturer's recommendation (Mirus Corporation, Madison, Wis.). For siRNA delivery, 10 μg pGL3 control, 1 μg pRL-SV40, and either 5 μg siRNA-Luc+ or 5 μg control siRNA were complexed with 16 μl TransIT™ In vivo in 2.5 ml total volume. Particles were injected over ˜7 s into the tail vein of 25-30 g ICR mice as described in Example 1. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA control. siRNA-luc+ specifically inhibited the target Luc+ expression 96% (Table 6).

TABLE 6
Delivery of siRNA to the mouse liver using TransIT ™ In
Vivo results in inhibition of target gene expression.

| | | | relative | % inhibition of |
|---|---|---|---|---|
| | | expression | LUC+ | Luc+ |
| complex | gene | (RLUs) | expression | expression |
| Plasmid alone | Luciferase | 31973057 | 5.1855 | 0.0 |
| | Renilla | 6165839 | | |
| Plasmid + | Luciferase | 853332 | 0.2069 | 96.0 |
| siRNA-Luc+ | Renilla | 4124726 | | |
| Plasmid + | Luciferase | 5152933 | 2.1987 | 57.5 |
| control SiRNA | Renilla | 2343673 | | |

These data show that the TransIT™ In Vivo labile polymer transfection reagent effectively delivers siRNA in vivo.

### Example 10

Physiological effects induced by siRNA delivery in vivo—Reduction of serum triglyceride levels using siRNA of HMG CoA reductase in vivo: We have demonstrated a reduction of serum triglyceride levels in mice upon treatment with siRNA directed against HMG CoA reductase. Group A (series2) mice (5 mice) were each injected with 50 μg of an siRNA directed against mouse HMG CoA reductase mRNA. Group B (Series1) mice (5 mice) were an uninjected control group. Group A and Group B animals were bled 7 days before, 2 days after, 4 days after, and 7 days after the injection. Serum samples were stored at −20° C. until all timepoints had been collected. Each group's serum samples from a given time-point were pooled prior to the triglyceride assays. Triglyceride assays were performed in quintuplicate.

Mice. Experiments were performed in Apoetm1Unc mice obtained from The Jackson Laboratories (Bar Harbor, Me.). Mice homozygous for the Apoetm1Unc mutation show a marked increase in total plasma cholesterol levels that is unaffected by age or sex. Fatty streaks in the proximal aorta are found at 3 months of age. The lesions increase with age and progress to lesions with less lipid but more elongated cells, typical of a more advanced stage of pre-atherosclerotic lesion. Moderately increased triglyceride levels have been reported in mice with this mutation on a mixed C57BL/6×129 genetic background.

siRNA reagents. Single-stranded, HMG CoA reductase-specific sense and antisense RNA oligomers with overhanging 3′ deoxyribonucleotides were ordered from Dharmacon, Inc. The annealed RNA duplex was resuspended in Buffer A (20 mM KCl, 6 mM HEPES-KOH pH 7.5, 0.2 mM MgCl₂) and stored at −20° C. prior to use. Prior to injection, siRNAs were diluted to the desired concentration (50 μg/2.2 ml) in Ringer's solution.

Oligonucleotide sequences. The sense oligomer with identity to the murine HMG CoA reductase gene has the sequence: 5′-rArCrArUrUrGrUrCrArCrUrGrCrUrArUrCrUrATT-3′ (SEQ ID 25), which corresponds to positions 2324-2344 of the HMG CoA reductase reading frame in the sense direction. The antisense oligomer with identity to the murine HMG CoA reductase gene has the sequence: 5′-rUrArGrArUrArG-rCrArGrUrGrArCrArArUrGrUTT-3′ (SEQ ID 26), which corresponds to positions 2324-2344 of the HMG CoA reductase reading frame in the antisense direction. The letter “r” preceding each nucleotide indicates that nucleotide is a ribonucleotide. The annealed oligomers containing HMG CoA reductase coding sequence are referred to as siRNA-HMGCR.

A total of 50 μg of siRNA-HMGCR was dissolved in 2.2 ml Ringer's solution (147 mM NaCl, 4 mM KCl, 1.13 mM CaCl₂), and injected into the tail vein of ApoE (−/−) mice over 7-12 seconds. Control mice were not injected and are referred to here as naive. Each mouse was bled from the retro-orbital sinus at various times prior to and after injection. Cells and clotting factors were pelleted from the blood to obtain serum. The serum triglyceride levels were then assayed by a enzymatic, colorimetric assay using the Infinity Triglyceride Reagent (Sigma Co.). Results showed that triglyceride levels in siRNA-HMGCR treated mice (Series2) were reduced 62% after two days, 56% after two days, and returned to normal levels after 7 days. No decrease in serum triglyceride levels was observed in uninjected mice (Series1).

Triglyceride assays. Serum samples were diluted 1:100 in the Infinity Triglyceride Reagent (2 μl in 200 μl) in a clear, 96-well plate. Each assay plate was then incubated at 37° C. for five minutes, removed and allowed to cool to room temperature. Absorbance was measured at 520 nm using a SpectraMax Plus plate reader (Molecular Devices, Inc). Background absorbance (no serum added) was subtracted from each reading and the resulted data was plotted versus timepoint.

TABLE 7
Triglyceride levels in animals following
delivery of HMGCR-specific siRNA

Triglyceride levels

| | | siRNA-HMGCR |
|---|---|---|
| day | No Injection Control | Treated Animals |
| −7 | 0.181 ± 0.010 | 0.218 ± 0.008 |
| +2 | 0.194 ± 0.011 | 0.082 ± 0.006 |
| +4 | 0.175 ± 0.012 | 0.095 ± 0.005 |
| +7 | 0.284 ± 0.021 | 0.189 ± 0.012 |

### Example 11

Physiological effects induced by siRNA delivery in vivo—Reduction of PPAR levels using siRNA expression cassettes in vivo: PPARα, peroxisome proliferator-activated receptor α, is a transcription factor and a member of the nuclear hormone receptor superfamily. The gene, found in both mice and humans, plays an important role in the regulation of mammalian metabolism. In particular, PPARα is required for the normal maintenance of metabolic pathways whose misregulation can facilitate the development metabolic disorders such as hyperlipidemia and diabetes. When bound to its ligand, PPARα binds to the retinoid X receptor (RXR) and activates the transcription of genes implicated in maintaining homeostatic levels of serum lipids and glucose. The manipulation of PPARα levels using RNA interference may be a safe and effective way to modulate mammalian metabolism and treat pathogenic hyperlipidemia and diabetes. We used a tail vein injection procedure to delivery plasmid DNA encoding an siRNA expression cassette to modulate endogenous PPARα levels using RNA interference in mice. Our results provide a model for the therapeutic delivery of siRNAs synthesized in vivo from delivered plasmid DNA. This method, or variations thereof, will be generally useful in the modulation of the levels of an endogenous gene using RNA interference.

siRNA hairpin sequences. Initially, we identified a series of plasmid DNA-based siRNA hairpins that exhibited RNAi activity against PPARα in primary cultured hepatocytes. The general hairpin structure consists of a polynucleotide sequence with sense and antisense target sequences flanking a micro-RNA hairpin loop structure. Transcription of the siRNA hairpin constructs was driven by the promoter from the human U6 gene. In addition, the end of the hairpin construct contains five T's to serve as an RNA Polymerase III termination sequence. The siRNA hairpin directed against PPARα had the sequence 5′-GGAGCTTT-GGGAAGAGGAAGGTGTCATCcttcctgtcaGATGGCATCTTCCTCTTCCCGAAGCTCC-TTTTT-3′ (SEQ ID 20). Lower-case letters indicate the sequence of the hairpin loop motif. The entire hairpin construct encoding the PPARα siRNA (consisting of the U6 promoter, the PPARα siRNA hairpin, and the termination sequence) is referred to as pMIR303. The negative control siRNA hairpin directed against GL3 had the sequence 5′-GGATTCCAA-TTCAGCGGGAGCCACCTGATgaagcttgATCGGGTGGCTCTCGCTGAGTTGGAATCC-ATTTTT-3′ (SEQ ID 21). The entire hairpin construct encoding the GL3 siRNA (consisting of the U6 promoter, the GL3 siRNA hairpin, and the termination sequence) is referred to as pMIR277.

Injections of mice. Ten mice in each experimental group were injected three times each with 40 μg/injection of either pMIR277 (GL3 siRNA construct) or pMIR303 (PPARα siRNA construct) using a tail vein injection procedure. Volumes of Ringer's solution (147 mM NaCl, 4 mM KCl, 1.13 mM CaCl₂) corresponding to 10% of each animal's body weight and containing the 40 μg of pMIR277 or pMIR303 were injected into mice over a period of 10 seconds with each injection. For each animal, injection 1 was performed on Day 0, injection 2 was performed on Day 2, and injection 3 was performed on Day 4. Seven days after Injection 3 (Day 11), livers from all mice were harvested and total RNA was isolated using the Tri-Reagent protocol.

Isolation of total RNA and cDNA synthesis. Total mRNA from injected mouse livers was isolated using Tri-Reagent. 500 ng of ethanol precipitated, total RNA suspended in RNase-free water was used to synthesize the first strand cDNA using SuperScript III reverse transcriptase. cDNAs were then diluted 1:50 and analyzed by quantitative, real-time qPCR.

Quantitative, real-time PCR. Bio-Rad's iCycler quantitative qPCR system was used to analyze the amplification of PPARα and GAPDH amplicons in real time. The intercalating agent SYBR Green was used to monitor the levels of the amplicons. Primer sequences used to amplify PPARα sequences were 5′-TCGGGATGTCACACAATGC-3′ (SEQ ID 30) and 5′-AGGCTTCGTGGATTCTCTTG-3′ (SEQ ID 16). Primer sequences used to amplify GAPDH sequences were 5′-CCTCTATATCCGTTTCCAGTC-3′ (SEQ ID 17) and 5′-TTGTCGGTGCAATAGTTCC-3′ (SEQ ID 31). Serial dilutions (1:20, 1:100 and 1:500) of cDNA made from Ringer's control samples were used to create the standard curve from which mRNA levels were determined. PPARα levels were quantitated relative to both GAPDH mRNA and total input RNA.

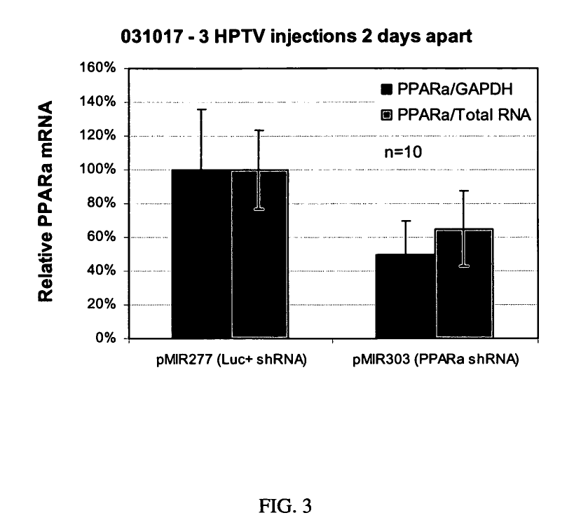

Results: Mouse livers injected with the PPARα hairpin constructs contained 50% or 35% less PPARα mRNA than those injected with GL3 siRNA control hairpins when compared to GAPDH mRNA or total input RNA, respectively. FIG. 3 shows the relative levels of PPARα mRNA as compared to GAPDH mRNA or total input RNA in each 10-mouse group. The experimental error is expressed as the total standard deviation among all samples. That this delivery procedure is able to achieve up to 50% knockdown of an endogenous target transcript demonstrates its general utility for in vivo modulation of gene expression.

### Example 12

Combination therapy using statins and siRNAs for the treatment of hyperlipidemia. Treatment with inhibitors of HMG CoA reductase, commonly known as statins, has been shown to markedly reduce the serum lipid levels of hyperlipidemia patients. Statins inhibit the activity of HMG-CoA reductase. In turn, this inhibition triggers a feedback mechanism through which the cellular levels of HMG-CoA reductase mRNA is markedly upregulated. Here, we present work that demonstrates a significant reduction in the levels of HMGCR mRNA in cells treated with atorvastatin. Addition of bioavailable siRNAs to the treatment regiments of patients on statins will lower the required statin dose, thereby reducing the required dosage of stains and cutting deleterious side effects.

siRNA reagents. Single-stranded, HMG CoA reductase-specific sense and antisense RNA oligomers with overhanging 3′ deoxyribonucleotides were ordered from Dharmacon, Inc. The annealed RNA duplex was resuspended in Buffer A (20 mM KCl, 6 mM HEPES-KOH pH 7.5, 0.2 mM MgCl₂) and stored at −20° C. prior to use. Prior to injection or transfection, siRNAs were diluted to the desired concentration (50 μg/2.2 ml) in Ringer's solution or (25 nM) in OPTI-MEM/Transit-TKO, respectively.

Oligonucleotide sequences. The sense oligomer with identity to the murine HMG CoA reductase gene has the sequence: SEQ ID 25, which corresponds to positions 2324-2344 of the HMG CoA reductase reading frame in the sense direction. The antisense oligomer with identity to the murine HMG CoA reductase gene has the sequence: SEQ ID 26, which corresponds to positions 2324-2344 of the HMG CoA reductase reading frame in the antisense direction. The letter “r” preceding each nucleotide indicates that nucleotide is a ribonucleotide. The annealed oligomers containing HMG CoA reductase coding sequence are referred to as siRNA-HMGCR.

A total of 50 μg of siRNA-HMGCR was dissolved in 2.2 ml Ringer's solution (147 mM NaCl, 4 mM KCl, 1.13 mM CaCl₂), and injected into the tail vein of mice over 7-120 seconds. Control mice were not injected and are referred to here as naive.

qPCR assays. Quantitative, real-time PCR was performed using the Bio-Rad iCycler system and iCycler reagents as recommended by the manufacturer. The primers used to amplify HMGCR sequences were SEQ ID 17 and SEQ ID 31.

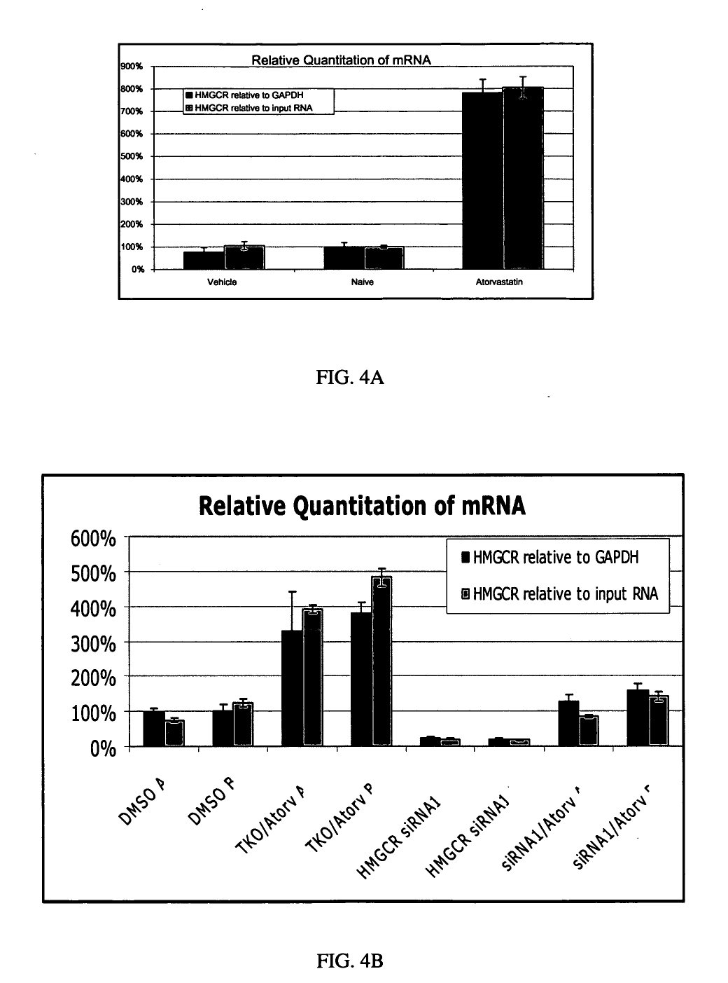

RESULTS Induction of HMG-CoA reductase in vivo. C57B6 mice treated for 48 hours with a 50 mg/kg dose of atorvastatin showed an expected and marked increase in HMG-CoA reductase mRNA levels as measured by quantitative, real-time PCR (FIG. 4A). Livers from groups of 10 mice were harvested 48 hours after treatment was commenced and pooled mRNA populations (10 mice/pool) were assayed for HMGCR levels. Mice treated with atorvastatin had, on average, an 800% increase in HMGCR mRNA.

Prevention of atorvastatin-induced upregulation of HMG-CoA reductase mRNA. As shown above, atorvastatin treatment results in a marked increase in the amount of HMGCR mRNA present in the livers of mice. Primary hepatocytes were isolated from C57B6 mice and cultured for 24 hours in the presence or absence of anti-HGMCR siRNAs and 10 μm atorvastatin. Total RNA from these cells was isolated and transcribed into cDNA using an oligo-dT primer and reverse transcriptase. Subsequently, HMGCR levels were assayed using quantitative, real-time PCR. HMGCR mRNA levels were induced 400% relative to vehicle-treated cells after 24 hours of exposure to atorvastatin (FIG. 4B). Simultaneous administration of the anti-HMGCR siRNA along with the statin held HMGCR levels to those seen in vehicle-treated controls. In addition, treatment of hepatocytes with the HMGCR-directed siRNA alone resulted in the knockdown of HMGCR mRNA to approximately 20% of that seen in control cells. These results show that the simultaneous delivery of an siRNA against HMGCR to cells treated with an HMGCR inhibitor can reduce the relative level of HMGCR mRNA to wild type levels seen in control cells. This strategy should reduce the amount of drug needed to inhibit cellular HMGCR and potentially lower the dose of drug needed in target validation or therapeutic applications in this and other protein families.

### Example 13

Combination therapy using statins and siRNAs for the treatment of hyperlipidemia in vivo. Initially, we identified a series of siRNAs that exhibited RNAi activity against PPARα in primary cultured hepatocytes. Having identified several highly active siRNAs, we selected one to use in our in vivo demonstration of siRNA delivery.

siRNA sequences. All RNA sequences were ordered from Dharmacon, Inc. The siRNA duplex directed against PPARα contained the target sequence 5′-rGrArTrCrGrGrArGrCrT-rGrCrArArGrArTrTrC-3′ (SEQ ID 28). A control GL3 siRNA duplex contained the target sequence 5′-rArArCrUrUrArCrGrCrUrGrArGrUrArCrUrUrCrGrA-3′ (SEQ ID 24). The “r” between each indicated base is used to indicate that the oligonucleotides are oligoribo-nucleotides. All siRNAs contained dTdT overhangs.

Injections of mice. Four mice in each experimental group were injected with 50 μg of siRNA using the high-pressure tail vein procedure. A volume of Ringer's solution (147 mM NaCl, 4 mM KCl, 1.13 mM CaCl₂) corresponding to 10% of each animal's body weight and containing 50 μg of PPARα siRNA sequences (or controls) were injected into mice over a period of 10 seconds. After 48 hours, livers from injected mice were harvested and total RNA was isolated.

Isolation of total RNA and cDNA synthesis. Total mRNA from injected mouse livers was isolated using Tri-Reagent. 500 ng of ethanol precipitated, total RNA suspended in RNase-free water was used to synthesize the first strand cDNA using SuperScript III reverse transcriptase. cDNAs were then diluted 1:50 and analyzed by quantitative, real-time qPCR.

Quantitative, real-time PCR. Bio-Rad's iCycler quantitative qPCR system was used to analyze the amplification of PPARα and GAPDH amplicons in real time. The intercalating agent SYBR Green was used to monitor the levels of the amplicons. Primer sequences used to amplify PPARα sequences were SEQ ID 30 and SEQ ID 16. Primer sequences used to amplify GAPDH sequences were SEQ ID 17 and SEQ ID 31. Primers used to amplify PTEN sequences were 5′-GGGAAGTAAGGACCAGAGAC-3′ (SEQ ID 23) and 5′-ATCATCTTGTGAAACAGCAGTG-3′ (SEQ ID 18). Serial dilutions (1:20, 1:100 and 1:500) of cDNA made from Ringer's control samples were used to create the standard curve from which mRNA levels were determined.

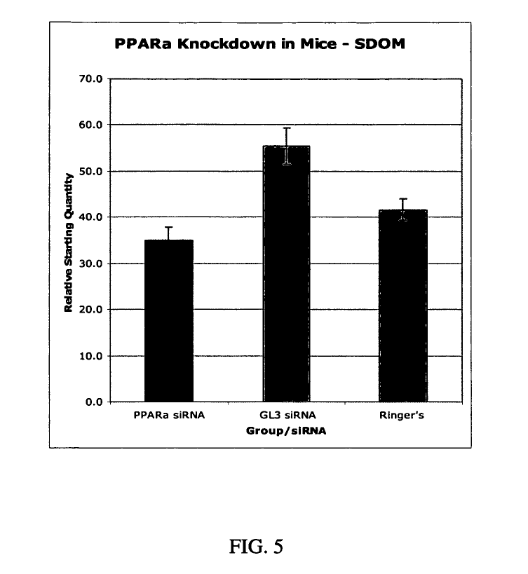

RESULTS: Mouse livers injected with siRNAs directed against PPARα contained 17% or 37% less PPARa mRNA than Ringer's control or GL3 siRNA control animals, respectively. FIG. 5 shows the relative levels of PPARa mRNA as compared to total input RNA in each four-mouse group. The experimental error is expressed as the standard deviation of the mean.

### Example 14

Combination treatment to reduce LDL-cholesterol levels in liver cells. Treatment with inhibitors of HMG-CoA Reductase, commonly known as statins, has been shown to markedly reduce serum LDL-cholesterol levels in hyperlipidemia patients. Statins inhibit the enzymatic activity of HMG-CoA Reductase. Inhibition of HMG-CoA Reductase causes decreased levels of cholesterol biosynthesis. To compensate for the reduced levels of cholesterol synthesis occurring in cells treated with statins, the low density lipoprotein receptor (LDLR) is upregulated through a specific, SREBP-dependent mechanism that senses the effective levels of cholesterol in cellular membranes. This upregulation of the LDLR results in increased cellular uptake of LDL-cholesterol and is one mechanism through which statins may exert their lipid-lowering effects. However, inhibition of cholesterol biosynthesis also triggers a feedback mechanism through which the cellular levels of HMG-CoA Reductase mRNA is markedly upregulated.

When HMG-CoA reductase activity drops below a certain threshold, the cell compensates by upregulating the LDL receptor, bringing cholesterol into the cell to replace the depleted endogenous stores. LDL receptor upregulation can be used as an indicator that HMG-CoA reductase activity had dropped below this threshold. We demonstrate that the levels of HMG-CoA reductase activity can be reduced by cotreatment with both statins and siRNA.

siRNA reagents. Single-stranded, HMG CoA reductase-specific sense and antisense RNA oligomers with overhanging 3′ deoxyribonucleotides were synthesized (Dharmacon, Inc). These single-stranded oligomers were annealed by stepwise cooling of a solution of the oligos from 96° C. to 15° C. The annealed RNA duplex was resuspended in Buffer A (20 mM KCl, 6 mM HEPES-KOH pH 7.5, 0.2 mM MgCl₂) and stored at −20° C. prior to use. Prior to transfection, siRNAs were diluted to the desired concentration (25 nM) in OPTI-MEM/TransIT-TKO (Mirus, Inc).

Oligonucleotide sequences. The antisense oligomer with identity to the murine HMG CoA reductase gene has the sequence: 5′-rCrCrArCrArArArUrGrArArGrArCrUrUrArUrATT-3′ (SEQ ID 27), which corresponds to positions 2793-2812 of the HMG CoA reductase reading frame in the sense direction. The antisense oligomer with identity to the murine HMG CoA reductase gene has the sequence: 5′-rUrArUrArArGrUrCrUrUrCrArUrUrUrGrUrGrGTT-3′ (SEQ ID 29), which corresponds to positions 2793-2812 of the HMG CoA reductase reading frame in the sense direction. The letter “r” preceding a nucleotide indicates that the nucleotide is a ribonucleotide. The annealed oligomers containing HMG CoA reductase coding sequence are referred to as siRNA-HMGCR.

Transfection and atorvastatin treatment of hepatocytes. Just prior to the addition of siRNA transfection cocktails (see below), fresh hepatocyte maintenance media supplemented with various concentrations of atorvastatin was added to each well in a 12-well, collagen coated plate that had been seeded with primary hepatocytes 24 hours previously. Then 100 μl of the siRNA transfection cocktail was added to each well. Hepatocyte maintenance media was a 1:1 mixture of DMEM-F 12/0.1 % BSA/0.1 % galactose.

siRNA transfection cocktail. Each 100 μl aliquot of siRNA transfection cocktail contained 3.8 μl TransIT-TKO, 275 nM siRNA, and the remaining volume of OPTI-MEM transfection media. The 100 μl aliquots were added to cells in 1 ml of media such that the final siRNA concentration was 25 nM.

RNA isolation. After 24 hours of siRNA transfection and atorvastatin treatment, cells were harvested in Tri-Reagent. RNA was isolated, quantitated, and corresponding cDNAs from an oligo-dT primer were synthesized with reverse transcriptase.

qPCR assays. Quantitative, real-time PCR was performed using the Bio-Rad iCycler system and iCycler reagents as recommended by the manufacturer. The primers used to amplify LDLR sequences were 5′-GCATCAGCTTGGACAAGGTGT-3′ (SEQ ID 19) and 5′-GGGAACAGCCACCATTGTTG-3′ (SEQ ID 22).

Primary hepatocytes were isolated from C57BL6 mice and plated on collagen-coated 12-well plates. After allowing them to adhere to the plates for 24 hours, one of two different procedures was followed. In the first, cells were treated with 200 nM atorvastatin in DMSO or DMSO alone for 24 hours. In the second, cells were covered with 1 ml of hepatocyte maintenance media. Next, 100 μl of an siRNA (HMGCR or GL3 control) cocktail (see above) was added to each well such that the final concentration of atorvastatin was 200 nM, 100 nM, 50 nM, 25 nM, or 0 nM and the final concentration of siRNA was 25 nM. Cells were incubated in the atorvastatin/siRNA mixture for 24 hours. Following all 24-hour incubations, cells were harvested in Tri-Reagent and processed for qPCR as described above.

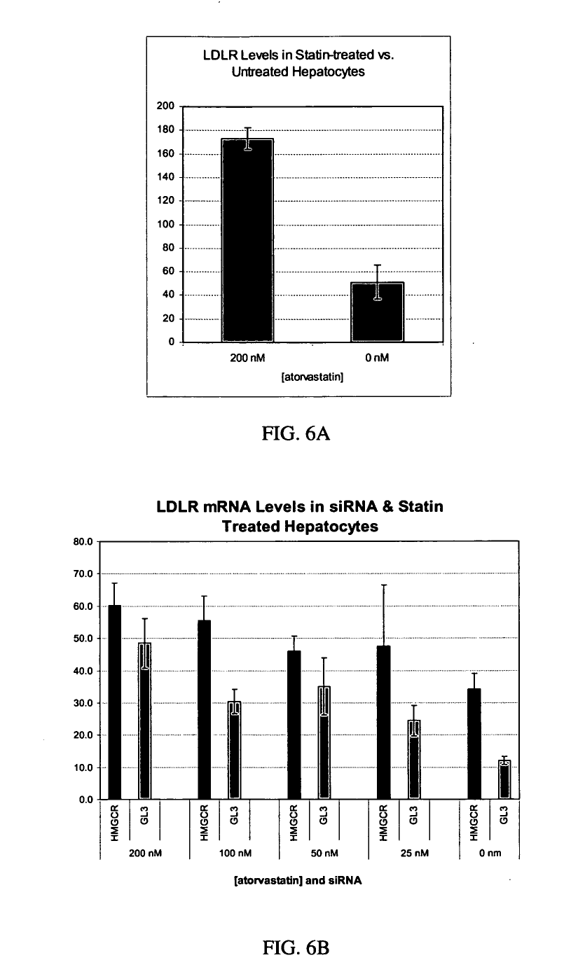

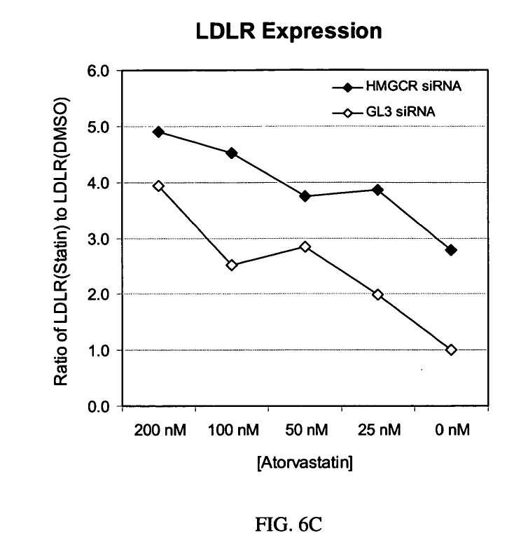

Induction of the LDL receptor in primary murine hepatocytes. Primary hepatocytes isolated by perfusion of C57BL6 mice and treated with 200 nM atorvastatin for 24 hours showed a marked increase in LDL receptor mRNA levels as measured by quantitative, real-time PCR (FIG. 6A).

This result shows the expected upregulation of LDLR mRNA upon treatment with atorvastatin. Next, we treated isolated hepatocytes with a range of atorvastatin concentrations and measured the amount of LDLR mRNA in each sample. In addition, cells were transfected with siRNAs against HMG-CoA reductase or control siRNA against luciferase (GL3). In each case, atorvastatin triggered a dose-dependent increase in LDLR mRNA levels (FIG. 6B). Furthermore, addition of siRNA to the cells further increased LDLR levels.

The data in FIG. 6B indicate that cells treated with HMGCR siRNAs required lower doses of atorvastatin to achieve a corresponding level of LDLR upregulation. For example, one can compare HMGCR siRNA-treated cells exposed to 25 nm or 50 nM statin with GL3 siRNA-treated cells exposed to 200 nM statin and see a similar level of LDLR mRNA was present in those cells. In addition, FIG. 6B indicates that simply reducing the amount of HMGCR in the cell results in an approximately 3-fold upregulation of LDLR mRNA (0 nM atorvastatin lanes). This shows that the HMGCR siRNA alone is effective in reducing cellular HMG-CoA reductase activity and thus increasing LDLR levels.

We used cells treated with GL3 siRNA and 0 nM atorvastatin as a baseline to compare the upregulation of LDLR mRNA in the other samples. The relative starting quantity of LDLR mRNA in each sample was plotted relative to the “baseline” LDLR mRNA level seen in GL3/no statin cells (FIG. 6C). The plot in FIG. 6C clearly shows that lower doses of atorvastatin were necessary to get comparable statin/no statin ratios in cells treated with HMGCR siRNAs.

In summary, we have demonstrated that siRNAs can be used to lower the effective dose of a small molecule inhibitor directed against the product of a gene targeted by the siRNA. This technology has applications in small molecule combination therapies as well as in drug discovery and research applications. For example, using siRNAs to decrease the gene dosage in cells being screened with small molecule libraries can sensitize cell-based assays and make otherwise difficult to detect cellular phenotypes apparent.

The principle demonstrated here can be applied to situations in which the target of the small molecule and the siRNA are not the same. For example, a small molecule inhibitor of a protein required for the efflux of cellular cholesterol (e.g., ABCA1), coupled with an siRNA against HMGCR mRNA, could work together to lower the levels of total serum cholesterol. This would be expected to result in the upregulation of the LDL receptor and a corresponding increase in LDL-C uptake. In addition, G-protein coupled receptor (GPCR) mediated signaling pathways could be modulated by simultaneously treating cells with GPCR antagonists and siRNAs targeting the second messenger pathways within cells.

### Example 15

Inhibition of Hepatitis B surface antigen (HBsAg) gene expression using siRNA in liver cells in vivo. The siRNAs used in this example were obtained from Dharmacon (Lafeyette, Colo.) and consisted of 21-nucleotide sense and antisense oligonucleotides each containing a two deoxynucleotide overhang at the 3′ end. The control siRNA targets positions 155-173 of the luc+ coding sequence: sense 5′-rCrUrUrArCrGrC-rUrGrArGrUrArCrUrUrCrGrATT-3′ (SEQ ID 4), antisense 5′-rUrCrGrArArGrUrArCrUrCrArGrCrGrUrArArGTT-3′(SEQ ID 5); HBsAg siRNA-1 targets positions 177-195 of the HBsAg coding sequence: sense 5′-rUrCrArCrUrCrArCrCrArArCrCrUrCrUrUrGrUTT-3′ (SEQ ID 8), antisense 5′-rArCrArArGrArGrGrUrUrGrGrUrGrArGrUrGrATT-3(SEQ ID 9). The letter “r” preceding a nucleotide indicates that nucleotide is a ribonucleotide. Sense and antisense strands for each siRNA, 40 μM each, were annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use.

Plasmid containing the HBsAg gene under the transcriptional control of the CMV enhancer promoter (pRc/CMV-HBs(S)) was obtained from Aldevron (Fargo, N.Dak.). Plasmid (pRc/CMV-HBs(S), 10 μg) was mixed with no siRNA, control siRNA (5 μg) or 0.5 or 5 μg of HepB sAg siRNA-1 and then diluted in 2 ml Ringer's solution (147 mM NaCl, 4 mM KCl, 1.13 mM CaCl₂) and injected into the tail vein of ICR mice (N=3) over 5-7 seconds. Serum was collected one day after injection. The amount of HBsAg in the serum was assayed by ELISA according to the manufacturer's instructions (Ortho) using purified HBsAg protein as the standard (Aldevron).

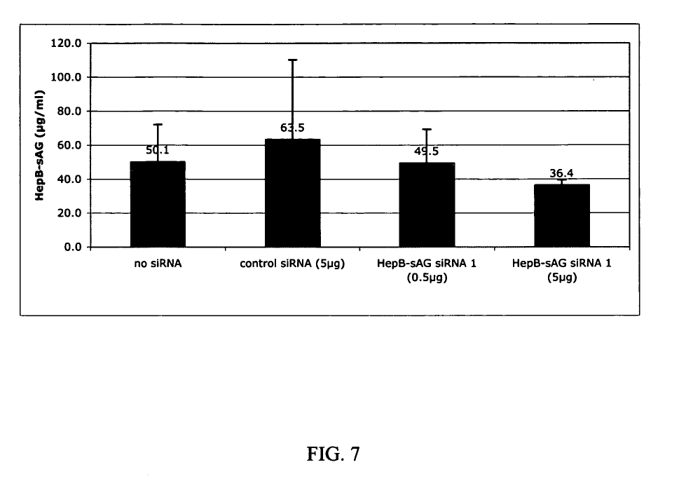

Co-injection of 10 μg pRc/CMV-HBs(S) and 0.5 μg HepB sAg siRNA-1 resulted in 22% inhibition of HBsAg expression compared to co-injection of 5 μg of the control siRNA (FIG. 7). Co-injection of 5 μg of HepB sAg siRNA-1 resulted in 43% inhibition of HBsAg expression as compared to co-injection of 5 μg of the control siRNA. These results indicate that RNAi can be used to inhibit expression of the HBsAg in liver cells in vivo.

### Example 16

Increased Inhibition of Hepatitis B surface antigen (HBsAg) gene expression using more potent siRNA in liver cells in vivo. The siRNA used in this example was obtained from Dharmacon (Lafeyette, Colo.) and consisted of 21-nucleotide sense and antisense oligonucleotides each containing a two deoxynucleotide overhang at the 3′ end. HBsAg siRNA-2 targets positions 392-410 of the HBsAg coding sequence:

sense:

(SEQ ID 14)
5′-rCrCrUrCrUrArUrGrUrArUrCrCrCrUrCrCrUrGTT-3′;

antisense:

(SEQ ID 15)
5′-rCrArGrGrArGrGrGrArUrArCrArUrArGrArGrGTT-3′;.

The letter “r” preceding a nucleotide indicates that nucleotide is a ribonucleotide. Sense and antisense strands for the siRNA, 40 μM each, were annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use.

Plasmid containing the HBsAg gene under the transcriptional control of the CMV enhancer promoter (pRc/CMV-HBs(S)) was obtained from Aldevron (Fargo, N.Dak.). Plasmid (pRc/CMV-HBs(S), 10 μg) was mixed with 5 μg of HBsAg siRNA-2 and then diluted in 2 ml Ringer's solution (147 mM NaCl, 4 mM KCl, 1.13 mM CaCl₂) and injected into the tail vein of ICR mice (N=3) over 5-7 seconds. Serum was collected one day after injection. The amount of HepB sAg in the serum was assayed by ELISA according to the manufacturer's instructions (Ortho) using purified HBsAg protein as the standard (Aldevron).

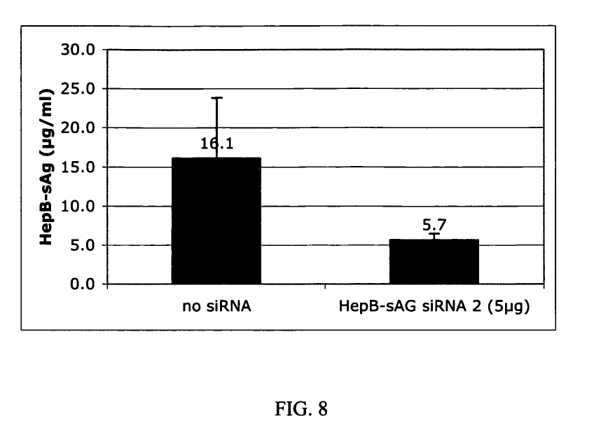

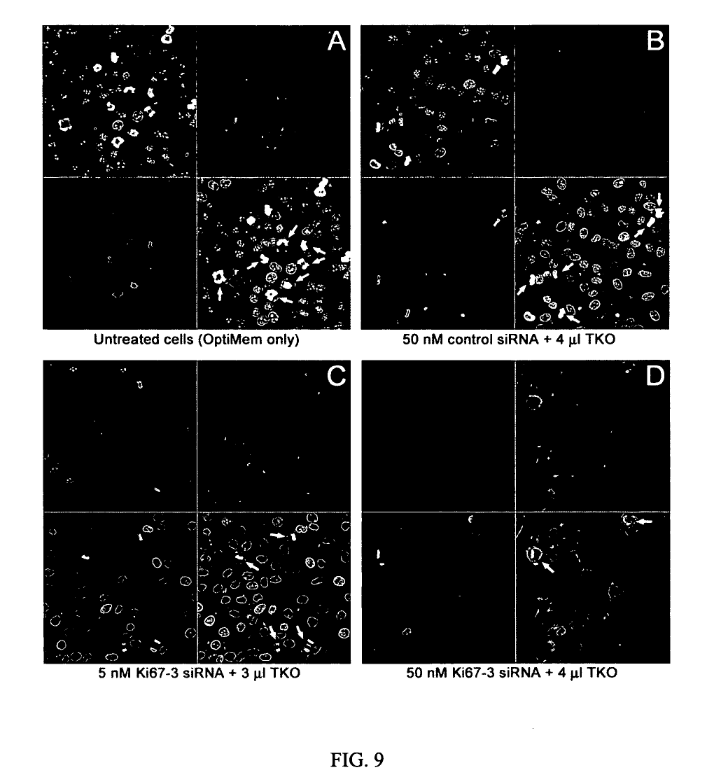

Co-injection of 10 μg pRc/CMV-HBs(S) and 5 μg HBsAg siRNA-2 resulted in 65% inhibition of HBsAg expression compared to injection of the plasmid DNA alone (FIG. 8).

### Example 17

Treatment of proliferative diseases, such as tumors, restenosis, angiogenic diseases, psoriasis, hypertrophic scars, ulcers etc. by using RNA interference to suppress the production of the Ki-67 protein. Ki-67 is a universally expressed protein involved in organizing the chromatin of cycling cells. Although any proliferative disease is a potential target for anti-Ki-67 based siRNA therapy, sites and diseases in which the proliferating cells are easily accessible for nucleic acid delivery are particularly attractive models. For example, diseases like psoriasis and restenosis can be targeted by the local administration of the therapeutic agent, without causing cell cycle effects in healthy cells. Although the expression of the Ki-67 antigen in cells involved in various proliferative diseases is not thought to be the cause of the disorder, down-regulating Ki-67 expression in these cells will inhibit proliferation of these cells. It has been shown that cell division can be suppressed by Ki-67-specific antisense oligonucleotides (Maeshima et al. 1996; Duchrow et al. 2001; Kausch et al. 2003; Kausch et al. 2004). We now demonstrate that a similar effect can be obtained by delivering to cells Ki-67-specific siRNA molecules. Consequently, alleviation of the symptoms of certain proliferative diseases by siRNA-based inhibition of Ki-67 expression is a potential therapeutic approach. Using processes standard in the art, the following Ki67-specific siRNAs were selected:

(SEQ ID 32)

MK167 #1: CGAGACGCCUGGUUACUAU

(SEQ ID 33)

MK167 #2: ACUCCAGUUGCCAGUGAUC

(SEQ ID 34)

MK167 #3: GACGGCAGUGUAUUAGAGA

(SEQ ID 35)

MK167 #4: GUGUAACUGGUAGCAAGAG

These siRNAs are complementary to sequences in Exon2 (MKI67 #1), Exon9 (MKI67 #2) and Exon13 (MKI67 #3 & 4) of the human Ki-67 mRNA. MKI67 #4 anneals to two 100% identical repeat sequence motifs in exon 13, thus lending it a higher chance to initiate mRNA cleavage.

The sequence of the firefly luciferase-specific siRNA that was used as a negative control for each experiment was: GL3: CUUACGCUGAGUACUUCGA (SEQ ID 2)

All siRNAs were synthesized as double stranded RNA fragments with 3′ dinucleotide dTdT overhangs.

6×10⁴ HeLa cells/well were plated into 24-well tissue culture plates. In an initial experiment, these cultures were treated with 5 or 50 nM final concentration of each siRNA, using TRANSIT-TKO® siRNA delivery reagent (Mirus Bio). Control cultures were either left untreated, treated with TransIT-TKO only, or transfected with a control siRNA (complementary to the firefly luciferase mRNA) at 50 nM final concentration. The complexes were formed in 50 μl serum-free OptiMEM medium, and were added to cells maintained in 250 μl DMEM supplemented with 10% FBS. 24 hours later cells from each well were trypsinized and split into new 24-well plates containing untreated, round glass coverslips (Electron Microscopy Sciences). The cultures were 5-fold diluted in order to give enough room for the cells to proliferate for 2 more days. 48 hrs later cells were fixed in 4% formaldehyde and permeabilized with 0.5% Triton-X-100 in PBS. Ki-67 expression was assessed by immunostaining using an anti-Ki-67 monoclonal antibody and a Cy3-labeled anti-MouseIgG F(ab′)₂ secondary antibody (Jackson ImmunoResearch). The cultures were also counterstained with 13 nM To-Pro-3 (DNA stain) and with 16.5 nM Alexa488-Phalloidin (actin stain; both from Molecular Probes).

MKI67 #3 siRNA was the most effective silencer and used for further studies to quantitate its inhibitory effect. MKI67 #3 was transfected into HeLa cells as described above, using 5 and 50 nM final concentration followed by splitting and diluting the cultures 24 hours later. The same controls were used as listed above. Transfection was repeated on the diluted cultures another 24 hours later, and the samples were processed 2 days later (on the 4th day after the original transfection). Immunostaining and counterstaining were performed as described above and images were collected by confocal microscopy, using identical settings for every sample. The total percent of Ki-67-positive cells (bright and dim), the percent of cells with bright Ki-67 signal, and the percent of cells in mitosis were determined for each sample.

To identify the fate of the cells in proliferation-inhibited cultures, an apoptosis assay was performed using the DeadEnd Fluorometric TUNEL Assay (Promega). The assay was run on cultures that were transfected twice with 25 or 50 nM MKI67 #3 siRNA, with 50 nM negative control siRNA (as above), with TransIT-TKO only or that were left untreated. Another untreated culture was used to DNase-treat the cells to create a positive control for the assay. Three sets of cultures were transfected identically and harvested 24, 48 or 72 hours after the second transfection. The cultures were processed according to the apoptosis kit manual and were finally counterstained with 13 nM To-Pro-3 DNA stain.

Results: Gene silencing efficiency of the four Ki-67-specific synthetic siRNAs. Initially, all 4 synthetic Ki-67 specific siRNAs were tested at 5 and 50 nM final concentrations by a single TransIT-TKO mediated transfection of HeLa cell cultures. All 4 sequences knocked down expression. The effect was evaluated by visual examination of microscopic images. At 5 nM final concentration only the MKI67 #3 siRNA sequence showed significant reduction in the intensity of the cells' Ki-67 signal. At 50 nM concentration all 4 sequences reduced expression, with #3 being the most effective. Cells treated with siRNA MKI67 #1 showed about 50% of the cells being Ki-67 negative. MKI67 #2 and #4 had weak silencing effect, leaving >50 % of the cells with bright Ki-67 signal even at 50 nM concentration.

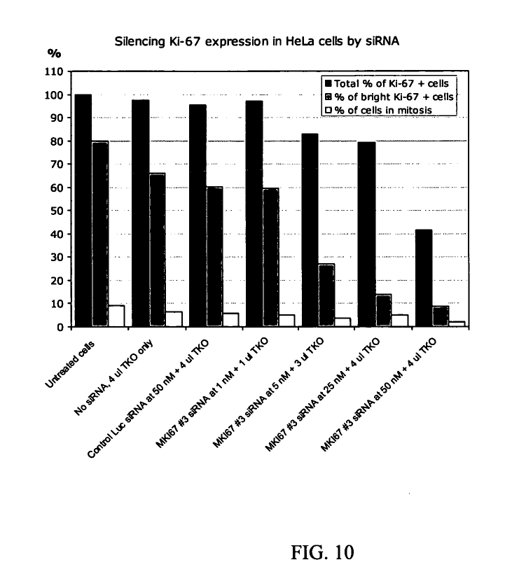

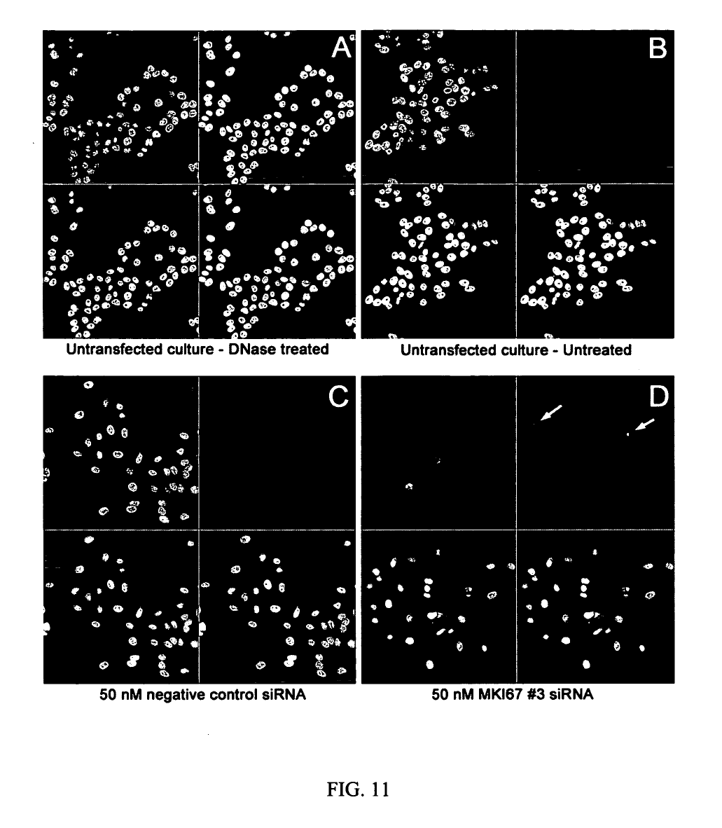

Quantitative evaluation of the silencing effect: In order to maximize gene silencing, the HeLa cell cultures were transfected twice with the MKI67 #3 siRNA using the same procedure, 48 hours apart. The cultures were split and diluted after the first 24 hours to maintain logarithmic growth phase. Two days after the second transfection the untreated control cultures were almost confluent, however, we could still detect a normal rate of mitotic activity (5-9%; FIG. 10). This suggested that the cultures were not contact-inhibited, providing a basis for a fair comparison of the mitotic activity of treated and untreated cultures. As FIG. 9 and FIG. 10 demonstrate, treatment with the MKI67 #3 siRNA caused dramatic knockdown in Ki-67 expression, and reduce mitotic activity about 50% (5-10% mitotic cells in the controls versus 2-5% in the siRNA treated cultures. The siRNA caused a dose-dependent decrease in the percent of total cells displaying Ki-67 immuno-staining: 95.5-100% in the controls, 97.2% at 1 nM, 83.1% at 5 nM, 79.1% at 25 nM and only 41.8% at 50 nM siRNA concentrations (FIG. 10). In the treated cultures most of these Ki-67 positive cells showed only a very faint signal, and the percent of cells with strong, bright signal diminished ever more when compared to the controls: 60.2-79.9% in the controls, 59.4% at 1 nM, 26.9% at 5 nM, 13.9% at 25 nM and a mere 8.8% at 50 nM MKI67 #3.

The visual examination of the cultures prior to fixation revealed a sizeable population of dead, floating cells that were removed by the PBS washes prior to fixation. The presence of dead cells and the lower final cell density observed in the siRNA-treated cultures (FIG. 9D versus FIGS. 9A and 9B) indicate that the siRNA treatment inhibited cell proliferation and/or cell viability.

In the untreated healthy cells, the Ki-67 antigen localized to the mitotic chromosomes and resulted in a very intense chromatin staining (FIG. 9A-B, arrows). In cells treated with 5 nM siRNA, this chromosome staining was still apparent, although significantly weaker than in the control (FIG. 9C, arrows). In the culture treated with 50 nM MKI67 #3, Ki-67 was almost undetectable on the condensed chromosomes (FIG. 9D, arrows). The lower cell density in the treated cultures indicates that overall mitotic activity was restricted. The overall cell density in treated cultures was approximately half of that of the untreated controls (FIG. 10D and FIG. 11D compared to FIG. 10A-B and FIG. 11A-B).

Conclusion: The MKI67 #3 siRNA is a potent inhibitor of Ki-67 expression in human cells. Since the Ki-67 protein is a crucial player in organizing the chromatin of proliferating cells, its absence results in decreased mitotic activity. While synthetic siRNA molecules were used for these experiments, it is possible to create an expression plasmid capable of long-term production of a hairpin or a short double stranded siRNA product.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A process for therapeutic treatment of hepatitis B in a mammal comprising:
a) making a polynucleotide-based gene expression inhibitor containing a sequence that is substantially complementary to a nucleic acid sequence in an expressed gene in said mammal;
b) inserting said polynucleotide-based gene expression inhibitor into a vessel in said mammal;
c) increasing the permeability of said vessel; and,
d) delivering said polynucleotide-based gene expression inhibitor to parenchymal cells in said mammal wherein said polynucleotide-based gene expression inhibitor is available to inhibit expression of said gene.

2. The process of claim 1 wherein said polynucleotide-based gene expression inhibitor consists of: an RNAi molecule, a small RNAi molecule, an siRNA and an miRNA.

3. The process of claim 1 wherein said polynucleotide-based gene expression inhibitor consists of an RNAi molecule expression vector.

4. The process of claim 1 further comprising: delivering to said mammal a small molecule drug.

5. The process of claim 4 wherein said small molecule drug and said polynucleotide-based gene expression inhibitor affect activity of a single gene

6. The process of claim 4 wherein said small molecule drug and said polynucleotide-based gene expression inhibitor affect activities of different genes.

7. The process of claim 1 wherein said gene is a viral gene.

8. The process of claim 7 wherein said viral gene encodes a structural component of said virus.

9. The process of claim 7 wherein said gene is involved in viral replication.

10. The process of claim 1 wherein said gene is an endogenous gene of said mammalian.

11. The process of claim 10 wherein inhibition of said gene reduces an immune response in said mammal.
